(19) United States
(12) Patent Application Publication
(10) Pub. No.: US 2025/0325465 A1
(43) Pub. Date: Oct. 23, 2025

(54) COSMETIC COLORING COMPOSITIONS CONTAINING MOISTURE CURABLE SILICONE POLYETHYLENEIMINE RESIN

(71) Applicant: HENKEL AG & CO. KGAA, Düsseldorf (DE)

(72) Inventors: Daniela DE LUCIA, Matawan, NJ (US); Sonny Yuxia LIU, Dayton, NJ (US); David ERLINGHEUSER, Wallingford, CT (US)

(21) Appl. No.: 18/873,344

(22) PCT Filed: Sep. 20, 2023
(86) PCT No.: PCT/US2023/074706

§ 371 Date: Dec. 10, 2024

Related U.S. Application Data
(60) Provisional application No. 63/371,589, filed on Aug. 16, 2022.
(60) Provisional application No. 63/371,591, filed on Aug. 16, 2022.

(51) Int. Cl. A61K 8/58; A61Q 5/06

(52) U.S. Cl. CPC A61K 8/585; A61Q 5/06; A61K 2800/43; A61K 2800/882

(57) ABSTRACT

A cosmetic composition for coloring keratin fibers includes a moisture-curable silicone resin formed from reaction of a silylated polyethyleneimine, an acid or a base, and at least one hydrolyzable silane having formula R¹ₙSiR²₄₋ₙ, wherein R¹ is alkyl, aryl, fluoroalkyl, trialkylsilyl, triarylsilyl, vinyl, or combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or combination thereof; n=0, 1, 2, or 3; R is alkyl or H. A coloring agent can be added to the moisture-curable silicone resin and the composition is applied to keratin fiber. A kit for coloring a keratin fiber contains a first container containing the moisture-curable silicone resin; and a second container containing coloring agent. The first container and the second container are mixed prior to application to the keratin fiber.

### CROSS-REFERENCE TO RELATED APPLICATIONS

The present application claims the benefit of U.S. Provisional Application Nos. 63/371,589 and 63/371,591, which were filed on Aug. 16, 2022, by which both are hereby incorporated by reference in their entirety.

### FIELD OF THE INVENTION

The present disclosure relates cosmetic compositions for treating keratin fibers, including human keratin fibers such as the hair, comprising moisture-curable silicone resins formed from the reaction of hydrolyzable silanes or siloxanes and silylated polyethyleneimine and at least one colorant. The disclosure also relates to a cosmetic process for treating keratin fibers.

### BACKGROUND OF THE INVENTION

The present invention relates to novel polyethyleneimine-silane and/or siloxane resins useful in the preparation of haircare compositions and, in particular, their use in dying or shaping compositions and other hair treatments for coloring or shaping keratin fibers without the need of damaging pre-treatments such as bleaching. In the field of dyeing keratin fibers, it is known practice to dye keratin fibers via various techniques using direct dyes for non-permanent colorations or dye precursors for permanent colorations. Those methods generally involve harsh pretreatments such as the use of oxidizing agents, i.e., H₂O₂ solutions, to remove fully or partly the natural hair pigmentation and to lift the cuticle to expose reactive groups on the hair surface that can then accommodate the selected dye or pigment. These processes are known to significantly weaken the hair fiber leading to breakage and, in extreme cases, to temporary hair loss.

Therefore, there is a need to develop innovative materials and cosmetic compositions for treating keratin fibers and for dyeing human keratin fibers, such as the hair, and for a hair dyeing process that can lead to colored coatings without the need to employ such harsh pre-treatments. In practice, dye films deposited on the hair, without such harsh pre-treatments, are susceptible to extraction by repeated shampooing, erosion by combing and brushing, and fading by exposure to sunlight and oxygen. Red colors are particularly susceptible to these degrading processes.

An object of the invention is to provide a new branched polyethyleneimine-silicone resin suitable in hair care compositions and methods to prepare said resin that will increase initial color uptake, provide resistance to erosion, abrasion, and chemical fading of the color. A still further objective is to provide compositions comprising said resin and a convenient kit that can be used by individual consumers to treat keratin fibers with the resin and compositions.

Organosilanes and organosiloxanes have been employed in cosmetic coloring compositions for keratin fibers. However, to achieve remanence they often require a multi-step application involving use of a primer composition followed by application of remaining components. Alternatively, or in addition, compositions will require application of heat to cure the materials, which can further damage fragile keratin fibers. Those known technologies have yet to achieve the lastingness or consistency of results that more common direct dyes technologies warrantee.

EP Patent No. 0705861 B1 discloses silylated polyamine polymers obtained by the exclusive use of linear polyethyleneimines, useful as lubricants. It does not claim branched polyethyleneimines; furthermore, the ratio described, and the combination of process and monomer ratio will result in a material that is not moisture curable.

U.S. Pat. No. 7,806,941 discloses compositions comprising silanes having one, two and three silicone atoms, at least one basic chemical function, such as primary, secondary, or tertiary amine, and at least one group chosen from hydroxyl and hydrolysable groups per molecule. The compositions also require at least one hydrophobic film forming polymer but do not contain polyethyleneimine. The examples use 80° C. heat to dry hair after application of treatments. Moreover, treatments are disclosed as “markedly inferior” unless a pretreatment containing aminoethoxysilanes is used.

U.S. Pat. No. 9,962,327 discloses sol/gel compositions for keratin materials, particularly nail varnish, which contain an alkoxysilane, an aminoalkoxysilane, and water in a specific amount related to the number of moles of alkoxy groups resulting from the alkoxysilane monomers or oligomers, and which may also contain dyes and/or colorant. These compositions also do not contain polyethyleneimine.

U.S. 2021/0290518 discloses compositions comprising organic carbon-based polymer, a linear or branched linking silicone, base compound, and a catalyst. The base compound is applied to keratin fibers as a pre-treatment. The organic polymer, linking silicone, and catalyst are applied and react in situ on the keratin fiber. The examples utilize isocyanate, which forms polyurethane/polyurea coatings on the keratin fiber.

It is desired to include polyethyleneimine (PEI) in keratin coatings without having to prime the keratin fibers with the polyethyleneimine. It is desired to have long lasting polyorganosilane or polyorganosiloxanes coatings for keratin fibers that can be applied in one step and/or do not require application of heat to cure. The present disclosure provides moisture-curable silicone resins containing silylated polyethyleneimine. By reacting silylated polyethyleneimine with certain organosilane or siloxane polymers prior to placement on keratin fibers, the inventors were able to provide durable silicone resins for coating keratin fibers that will cure when applied to keratinous material and exposed to ambient moisture. When color is included with such silicone polyethyleneimine resins, they provide resistance to erosion, abrasion, and chemical fading of the color.

It was unexpectedly found that reacting silylated polyethyleneimine, such as silylated branched polyethyleneimine, with certain silanes and/or siloxanes prior to application to keratin can remove the need for particular relative humidity to make the compositions suitable for coating the keratin fibers and/or for use of an aminosilicone pretreatment to achieve proper adhesion. The silylated polyethyleneimines can be formed in situ in a single pot reaction with additional organosilane/siloxane components or can be separately formed and added to react. Regardless, the incorporation of the silylated polyethyleneimine into a silicone resin is performed prior to application to keratin fiber.

The use of branched-PEI (BPEI) is well known to help condensation between silanol groups in the adhesives industry but is not typically used because it is difficult to remove post reaction. However, advantageously, it was discovered that chemically bonding BPEI in a silicone resin makes it difficult to leach out and eliminates the need to remove it after reaction.

The disclosed silicone resins can be easily mixed with colorants and dyes and are able to cure when exposed to ambient moisture. As such, the resin can be applied to keratin fibers and provide a durable coating within minutes to hours after being applied to dry or wet keratin fiber. This advantageously avoids any preparation of the fibers, application of peroxides or oxidative catalysts, curing agents, and/or application of heat and preserves the integrity of the fiber, simplifies the keratin treatment process, and eliminates user errors.

### SUMMARY OF THE INVENTION

In one aspect, the present disclosure provides a moisture-curable silicone resin formed from reaction of
a silylated polyethyleneimine;
at least one hydrolyzable silane having formula R¹ₙSiR²₄₋ₙ,
   wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H;
   and
an acid or a base.

The resulting resins will have at least one amino functional group of the silylated polyethyleneimine covalently bonded with a hydrolyzable silane group —SiR²ₙ, wherein n=1, 2, or 3. Advantageously, the resins cure at ambient conditions within several minutes to several hours.

In some embodiments, the at least one hydrolyzable silane having formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof. In certain embodiments, the at least one hydrolyzable silane having formula R¹ₙSiR²₄₋ₙ is methyltriethoxysilane, tetraethoxysilane, diethoxydimethylsilane, n-octyltriethoxysilane, 1,8-bis(triethoxysilyl)octane, or a combination thereof, most preferably methyltriethoxysilane.

In certain embodiments, the reaction forming the moisture-curable silicone resin further comprises a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H. In certain of those embodiments, the hydrolyzable aminosilane having formula R³ₙSiR⁴₄₋ₙ is selected from the group consisting of 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof. In certain preferred embodiments, the hydrolyzable aminosilane is 3-aminopropyltriethoxysilane (AMEO).

In some embodiments, the silylated polyethyleneimine is a reaction product of polyethyleneimine and a hydrolyzable silane having formula R⁵ₙSiR⁶₄₋ₙ,
wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H.

In some of those embodiments, a molar ratio of the hydrolyzable silane having formula R⁵ₙSiR⁶₄₋ₙ to the polyethyleneimine is from about 1:100 to about 100:1, more preferably 1:1 to 1:10, most preferably about 1:3 to 1:5. In some embodiments, R⁵ is epoxyalkyl, (meth)acryloxyalkyl, or halogenalkyl, such as in 3-glycidoxypropyltriethoxysilane, 3-(meth)acryloxypropyltriethoxysilane, or 3-chloropropyltriethoxysilane. In some embodiments, the hydrolyzable silane having formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof, most preferably 3-glycidoxypropyltrimethoxysilane.

In certain preferred embodiments, the silylated polyethyleneimine comprises linear, branched or dendrimer polyethyleneimine, preferably branched. In certain embodiments, the polyethyleneimine is linear, branched, or dendrimer polyethyleneimine having a molecular weight ranging between 300 and 100,000.

In certain embodiments the acid or base is a Lewis acid or base or a Bronsted acid or base. In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

The reaction to form the resin may contain a solvent, such as alcohol, water, aliphatic hydrocarbons,-aromatic hydrocarbons, ether, tetrahydrofuran, ethyl acetate, methyl ethyl ketone, or a mixture thereof.

The resins will typically have a molecular weight of 400 g/mol to 2,000,000 g/mol.

The resins can be included in compositions that can contain at least one cosmetically or dermatologically active agent, selected from the group consisting of plasticizers, vitamins, fragrances, trace elements, softeners, plasticizers, coalescers, preserving agents, stabilizers, co-resins, anti-foams, and spreading agent and/or may be combined with a coloring agent. The resins and compositions containing them can be applied as a coating, sealant, adhesive, or varnish, typically on keratinous materials. The compositions are useful for haircare, hair coloring, nail varnish or varnish base.

Also disclosed is a moisture-curable silicone resin formed from reaction of
at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3;
a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3;
   a hydrolyzable silane having formula R⁵ₙSiR⁶₄₋ₙ, wherein R⁵ is epoxyalkyl, (meth)acryloxyalkyl, acryloxyalkyl, haloalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3;
   polyethyleneimine; and
   an acid or a base.

In some embodiments, the at least one hydrolyzable silane having formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof. In some of those embodiments, the first hydrolyzable silane having formula R¹ₙSiR²₄₋ₙ is methyltriethoxysilane, triethoxymethylsilane, diethoxydimethylsilane, n-octyltriethyoxysilane, 1,8-bis(triethoxysilyl)octane, or a combination thereof, most preferably methyltriethoxysilane.

In certain embodiments, the hydrolyzable aminosilane having formula R³ₙSiR⁴₄₋ₙ is selected from the group consisting of 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof. In certain preferred embodiments, the hydrolyzable aminosilane is 3-aminopropyltriethoxysilane (AMEO).

In some embodiments, a molar ratio of the hydrolyzable silane having formula R⁵ₙSiR⁶₄₋ₙ to the polyethyleneimine is from about 1:100 to about 100:1, more preferably about 1:1 to 1:10, most preferably about 1:3 to about 1:5. In some embodiments, R⁵ is epoxyalkyl, (meth)acryloxyalkyl, or halogenalkyl, such as in 3-glycidoxypropyltriethoxysilane, or 3-chloropropyltriethoxysilane. In some embodiments, the hydrolyzable silane having formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof, most preferably 3-glycidoxypropyltrimethoxysilane.

In certain preferred embodiments, the polyethyleneimine is linear, branched, or a dendrimer polyethyleneimine having a molecular weight ranging between 300 and 100,000. Preferably, the polyethyleneimine is branched.

In certain embodiments the acid or base is a Lewis acid or base or a Bronsted acid or base. In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

The reaction to form the resin may contain a solvent, such as alcohol, water, aliphatic hydrocarbons,-aromatic hydrocarbons, ether, tetrahydrofuran, ethyl acetate, methyl ethyl ketone, or a mixture thereof.

The resins will typically have a molecular weight of 400 g/mol to 2,000,000 g/mol.

The resins can be included in compositions that can contain at least one cosmetically or dermatologically active agent, selected from the group consisting of plasticizers, vitamins, fragrances, trace elements, softeners, plasticizers, coalescers, preserving agents, stabilizers, co-resins, anti-foams, and spreading agent and/or may be combined with a coloring agent. The resins and compositions containing them can be applied as a coating, sealant, adhesive, or varnish, typically on keratinous materials. The compositions are useful for haircare, hair coloring, nail varnish or varnish base.

In another aspect, a kit for coloring a keratin fiber is provided. The kit comprises a first composition comprising a moisture-curable silicone resin disclosed herein; and a second composition comprising at least one coloring agent selected from pigments, direct dyes, or a mixture thereof. The first composition and the second composition are mixed prior to application to the keratin fiber. In certain embodiments, the second composition can further comprise a film forming polymer, and optionally a surfactant, an acid or a base, water and/or a solvent. The kit optionally contains a third container comprising a sealer, which can be a UV curable sealer comprising at least one photoinitiatior and at least one acrylate monomer or oligomer. The photoinitiator can be ethyl (2,4,6-trimethylbenzoyl) phenylphosphinate, diphenyl(2,4,6-trimethylbenzoyl)phosphine oxide, phenylbis(2,4,6-trimethylbenzoyl)phosphine oxide, or mixture thereof. The acrylate monomer or oligomer may be a multifunctional acrylate having a molecular weight between 200 and 10,000 Daltons.

In yet another aspect, the disclosure provides a process for preparing a moisture-curable silicone resin comprising
mixing at a temperature of from 0° C. to 100° C. for 0.1 to 12 hours water, an acid or a base, and at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H;
   and
adding silylated polyethyleneimine and a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H,
   wherein at least one amino-functional group of the silylated polyethyleneimine is covalently bonded with a hydrolyzable silyl group —SiR²ₙ, wherein n=1, 2 or 3; or a hydrolysable silyl group —X—SiR⁵ₙR⁶₄₋ₙ, wherein R⁵ is epoxyalkyl, methacryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=0, 1, or 2; R is alkyl or H; X is alkylene.

In some embodiments, the reaction is mixed at a temperature of about 10-50° C., more preferably about 20-40° C. In certain embodiments, after adding the silylated polyethyleneimine, the reaction is mixed at about 80-200° C. In certain of those embodiments, mixing occurs for 1 minute to 6 hours, or 5 minutes to 3 hours, or 10 minutes to 1.5 hours, or about 20 to 40 minutes.

In certain embodiments the acid or base is a Lewis acid or base or a Bronsted acid or base. In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

In certain embodiments, the at least one hydrolyzable silane having formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof. In some of those embodiments, the at least one hydrolyzable silane is methyltriethoxysilane, triethoxymethylsilane, diethoxydimethylsilane, n-octyltriethyoxysilane, 1,8-bis(triethoxysilyl)octane, or a combination thereof, most preferably methyltriethoxysilane.

In some embodiments, the silylated polyethyleneimine is a reaction product of polyethyleneimine and a hydrolyzable silane having formula R⁵ₙSiR⁶₄₋ₙ, wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H, and at least one amino-functional group of the silylated polyethyleneimine is covalently bonded with a hydrolyzable silyl group —X—SiR⁵ₙR⁶₄₋ₙ.

In some of those embodiments, the polyethyleneimine and the hydrolyzable silane having formula R⁵ₙSiR⁶₄₋ₙ are mixed at a temperature of 20-200° C. for at least 1 hour. In certain of those embodiments, the polyethyleneimine and the hydrolyzable silane having formula R⁵ₙSiR⁶₄₋ₙ are mixed at about 50-200° C., more preferably about 70-120° C., most preferably about 80-95° C. for at least 30 minutes, more preferably at least 45 minutes, most preferably about 1 hour to 3 hours.

In certain embodiments, a molar ratio of the hydrolyzable silane having formula R⁵ₙSiR⁶₄₋ₙ to the polyethyleneimine is from about 1:100 to about 100:1, more preferably about 1:1 to about 1:10, most preferably about 1:3 to about 1:5.

In some embodiments, R⁵ is epoxyalkyl, (meth)acryloxyalkyl, or halogenalkyl, such as in 3-glycidoxypropyltriethoxysilane, or 3-chloropropyltriethoxysilane. In some embodiments, the hydrolyzable silane having formula R⁵ₙSiR⁶₄₋ₙ is selected from the group consisting of 3-methacryloxypropyltriethoxysilane 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof, most preferably 3-glycidoxypropyltriethoxysilane.

In certain embodiments, the polyethyleneimine is linear, branched, or dendrimer polyethyleneimine having a molecular weight ranging between 300 and 100,000. In certain preferred embodiments, the silylated polyethyleneimine comprises branched polyethyleneimine.

In some embodiments, the hydrolyzable aminosilane having formula R³ₙSiR⁴₄₋ₙ is selected from the group consisting of 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof. In certain preferred embodiments, the hydrolyzable aminosilane having formula R³ₙSiR⁴₄₋ₙ is 3-aminopropyltriethoxysilane (AMEO).

In certain embodiments, after adding aminosilane, the reaction is allowed to proceed at a temperature of about 10-50° C., more preferably about 20-40° C. In certain embodiments, the reaction is mixed at about 60° C. to about 200° C., or about 70° C. to about 120° C., or about 80° C. to about 95° C. In certain embodiments, mixing occurs for 30 minutes to 6 hours, or about 1 hour to about 3 hours.

The reaction to form the resin may contain additional solvent, such as alcohol, aliphatic hydrocarbons,-aromatic hydrocarbons, ether, tetrahydrofuran, ethyl acetate, methyl ethyl ketone, or a mixture thereof.

Another process for preparing a moisture-curable silicone resin comprises
(i) Mixing e.g., stirring at a temperature of from 0° C. to 100° C. for 0.1 to 12 hours
a. water,
b. an acid or a base,
c. at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H; and
d. a hydrolyzable silane having formula R⁵ₙSiR⁶₄₋ₙ, wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H;
(ii) adding polyethyleneimine to the mixture of step (i) and mixing at a temperature of from 0° C. to 100° C. for 0.1 to 12 hours;
(iii) optionally, adding to step (ii) a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H; and
(iv) heating for at least 30 minutes.

In some embodiments, the (i) mixing at a temperature of from 0° C. to 100° C. occurs at about 10-50° C., most preferably about 20-40° C. In certain embodiments, mixing of step (i) occurs for 1 minute to 6 hours, more preferably 5 minutes to 3 hours, more preferably 10 minutes to 1.5 hours, most preferably about 20 to 45 minutes. In certain embodiments, the mixing comprises or consists of stirring.

In certain embodiments the acid or base is a Lewis acid or base or a Bronsted acid or base. In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

In certain embodiments, the at least one hydrolyzable silane having formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof. In some of those embodiments, the at least one hydrolyzable silane is triethoxymethylsilane, tetraethoxysilane, diethoxydimethylsilane, n-octyltriethoxysilane, 1,8-bis(triethoxysilyl)octane, or a combination thereof, most preferably methyltriethoxysilane.

In some embodiments, R⁵ of the hydrolyzable silane having formula R⁵ₙSiR⁶₄₋ₙ is epoxyalkyl, (meth)acryloxyalkyl, or halogenalkyl. In some embodiments, the hydrolyzable silane having formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof, most preferably 3-glycidoxypropyltrimethoxysilane.

In certain embodiments, the polyethyleneimine is linear, branched, or dendrimer polyethyleneimine having a molecular weight ranging between 300 and 100,000. In certain preferred embodiments, the silylated polyethyleneimine comprises branched polyethyleneimine.

In some embodiments, a molar ratio of the (d) hydrolyzable silane having formula R⁵ₙSiR⁶₄₋ₙ to the polyethyleneimine is from about 1:100 to about 100:1, more preferably about 1:1 to about 1:10, most preferably about 1:3 to about 1:5.

In some embodiments, the mixing of step (ii) occurs for 5 minutes to 3 hours, more preferably 10 minutes to 1.5 hours, most preferably about 20 to 40 minutes. The mixing of step (ii) can occur from 0° C. to 100° C., more preferably at about 10-50° C., most preferably about 20-40° C. In certain embodiments, mixing of step (ii) occurs for 1 minute to 6 hours, more preferably 5 minutes to 3 hours, more preferably 10 minutes to 1.5 hours, most preferably about 20 to 40 minutes.

In certain embodiments, the step (iii) hydrolyzable aminosilane having formula R³ₙSiR⁴₄₋ₙ is selected from the group consisting of -aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof. In certain preferred embodiments, the hydrolyzable aminosilane is 3-aminopropyltriethoxysilane (AMEO).

In some embodiments, the heating is to about 50-200° C., more preferably about 70-120° C., most preferably about 80-95° C. for at least 45 minutes, most preferably about 1 hour to 3 hours.

The reaction to form the resin may contain additional solvent, such as alcohol, aliphatic hydrocarbons,-aromatic hydrocarbons, ether, tetrahydrofuran, ethyl acetate, methyl ethyl ketone, or a mixture thereof.

In a further aspect, a cosmetic composition for coloring keratin fibers comprises a moisture-curable silicone resin formed from reaction of a silylated polyethyleneimine, an acid or a base, and at least one hydrolyzable silane having formula R¹ₙSiR²₄₋ₙ,
wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H;
   and
   at least one coloring agent selected from pigments, direct dyes, or a mixture thereof. At least one amino functional group of the silylated polyethyleneimine is covalently bonded with a hydrolyzable silane group —SiR²ₙ, wherein n=1, 2 or 3; or —X—SiR⁵ₙR⁶₄₋ₙ, wherein n=0, 1, or 2. A kit for coloring a keratin fiber can comprise a first composition comprising the moisture-curable silicone resin and a second composition comprising the at least one coloring agent selected from pigments, direct dyes, or a mixture thereof. The content of the first composition and the second composition are mixed into a ready-to-use silicone resin color composition prior to application to the keratin fiber. The second composition can further comprise a film forming polymer, optionally a surfactant, an acid or a base, water and/or a solvent. In some embodiments, the at least one coloring agent comprises about 0.5% to about 10% by weight, preferably about 1-5% by weight of the first composition. The ready-to-use silicone resin color composition preferably comprises about 5% to about 20%, preferably about 10% by weight of the silicone resin and about 0.5% to about 2% by weight of coloring agent.

In some embodiments, the at least one hydrolyzable silane having formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, or a combination thereof, most preferably methyltriethoxysilane.

In certain embodiments the acid or base is a Lewis acid or base or a Bronsted acid or base. In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

In some embodiments, the reaction forming the moisture-curable silicone resin further comprises a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H.

In certain embodiments, the hydrolyzable aminosilane is selected from the group consisting of 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof. Preferably, the hydrolyzable aminosilane is 3-aminopropyltriethoxysilane (AMEO).

In some embodiments, the silylated polyethyleneimine is a reaction product of polyethyleneimine and a hydrolyzable silane having formula R⁵ₙSiR⁶₄₋ₙ, wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H, and at least one amino-functional group of the silylated polyethyleneimine is covalently bonded with a hydrolyzable silyl group —X—SiR⁵ₙR⁶₄₋ₙ, wherein n=0, 1, or 2. In some of those embodiments, a molar ratio of the hydrolyzable silane having formula R⁵ₙSiR⁶₄₋ₙ to the polyethyleneimine is from about 1:100 to about 100:1. In some embodiments, R⁵ is epoxyalkyl, (meth)acryloxyalkyl, or halogenalkyl, such as in 3-glycidoxypropyltriethoxysilane, or 3-chloropropyltriethoxysilane. In some embodiments, the hydrolyzable silane having formula R⁵ₙSiR⁶₄₋ₙ is selected from the group consisting of 3-methacryloxypropyltriethoxysilane 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof, preferably 3-glycidoxypropyltriethoxysilane. The polyethyleneimine can be linear, branched, or dendrimer polyethyleneimine having a molecular weight ranging between 300 and 100,000. In certain preferred embodiments, the silylated polyethyleneimine comprises branched polyethyleneimine.

In certain embodiments, the cosmetic composition further comprises at least one agent selected from the group consisting of plasticizers, vitamins, fragrances, trace elements, softeners, plasticizers, coalescers, preserving agents, stabilizers, co-resins, anti-foams, spreading agents. In preferable embodiments, the moisture-curable silicone resin comprises about 5% to about 20%, preferably about 10% by weight of the silicone resin. In certain embodiments, the cosmetic composition further comprises at least one coloring agent selected from pigments, direct dyes, or a mixture thereof. In some of those embodiments, the at least one coloring agent comprises about 0.5% to about 10% by weight, preferably about 1-5%, more preferably about 0.5% to about 2% by weight of the cosmetic composition.

In one particular aspect, a cosmetic composition for coloring keratin fibers comprises
(i) a moisture-curable silicone resin formed from reaction of
(a) at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3;
(b) a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3;
(c) a hydrolyzable silane having formula R⁵ₙSiR⁶₄₋ₙ, wherein R⁵ is epoxyalkyl, (meth)acryloxyalkyl, acryloxyalkyl, haloalkyl, isocyanatoalkyl or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3;
(d) polyethyleneimine; and
(e) an acid or a base;
   and
(ii) at least one coloring agent selected from pigments, direct dyes, or a mixture thereof.
   The moisture-curable silicone resin is formed prior to application on keratinous materials.

The reaction to form the resin may contain a solvent, such as alcohol, water, aliphatic hydrocarbons,-aromatic hydrocarbons, ether, tetrahydrofuran, ethyl acetate, methyl ethyl ketone, or a mixture thereof.

The polyethyleneimine can be linear, branched, or a dendrimer polyethyleneimine having a molecular weight ranging between 300 and 100,000. In preferred embodiments, a molar ratio of the hydrolyzable silane having formula R⁵ₙSiR⁶₄₋ₙ to the polyethyleneimine is from about 1:100 to about 100:1. In preferable embodiments, the moisture-curable silicone resin comprises about 1% to about 20%, preferably about 2.5% to about 15%, more preferably about 5-10% by weight of the cosmetic composition. In certain embodiments, the at least one coloring agent comprises about 0.5% to about 10% by weight, preferably about 1-5%, more preferably about 0.5% to about 2% by weight of the cosmetic composition. In some embodiments, the cosmetic composition further comprises at least one agent selected from the group consisting of plasticizers, vitamins, fragrances, trace elements, softeners, plasticizers, coalescers, preserving agents, stabilizers, co-resins, anti-foams, spreading agents. A kit for coloring a keratin fiber can comprise a first composition comprising the moisture-curable silicone resin and a second composition comprising the at least one coloring agent selected from pigments, direct dyes, or a mixture thereof. The first composition and the second composition are mixed prior to application to the keratin fiber. The second composition can further comprise a film forming polymer, optionally a surfactant, an acid or a base, water and/or a solvent.

A method of coloring keratin fibers comprises applying the cosmetic compositions described herein to keratin fibers, preferably dry keratin fibers, and exposing the fibers to ambient conditions without rinsing or without application of heat for 5 minutes to 48 hours. As mentioned, the moisture-curable silicone resins are formed prior to application on keratinous materials and can simply cure by exposure to ambient humidity. In certain embodiments, the fibers are exposed to ambient conditions without rinsing or without application of heat for 5 minutes to 3 hours. In some of those embodiments, the method further comprises applying a sealer comprising acrylic polymer and/or cellulose for 1 to 45 minutes, most preferably from 1 to 15 minutes. The sealer can be a UV curable sealer comprising at least one photoinitiator and at least one acrylate monomer or oligomer. The photoinitiator can be ethyl (2,4,6-trimethylbenzoyl) phenylphosphinate, diphenyl(2,4,6-trimethylbenzoyl)phosphine oxide, phenylbis(2,4,6-trimethylbenzoyl)phosphine oxide, or mixture thereof. The acrylate monomer or oligomer may be a multifunctional acrylate having a molecular weight between 200 and 10,000 Daltons. The sealer may be cured with UV light or heat. In certain of those embodiments, the sealed hair is further rinsed and dried. Advantageously, color remanence is achieved for up to 20 washes regardless of whether a sealer is applied.

### BRIEF DESCRIPTION OF THE DRAWINGS

FIG. 1 shows images of the resins of Examples 1, 15 and 16 after curing at ambient conditions.

FIG. 2 is IR spectra of the product of Example 4 against the starting materials.

FIG. 3 shows images of hair strands in accordance with Example 22 that have been pre-treated with or without sebum followed by application of silicone resin color gel 37A of Example 21 (no BPEI) 48 hours after application.

FIG. 4 shows the strands of FIG. 3 after 20 washing cycles performed 48 hours after application.

FIG. 5 shows a compilation of rub-off test results of Example 22 for each timepoint (1 hr., 24 hrs., 48 hrs.) when silicone resin color gels 38 (left column containing BPEI) and 37A (right column, no BPEI) were applied to dry hair.

FIG. 6 shows images from the rub off test of Example 22 in which color gel 38A of Example 21 has been applied to either dry (left) or wet (right) hair pre-treated with sebum at 48 hours after application when the strands were stored at 45% RH.

FIG. 7 shows images comparing hair strands to which color gel 38A containing BPEI was applied to hair strands pre-treated with sebum to control 37A (no BPEI). For low humidity (“LH”) samples, the color gels were applied to dry or wet hair strands and maintained at 16% RH. For high humidity (“CTH”) samples, the color gels 38A and 37A were applied to wet hair strands and maintained at 45% RH. The left column shows the strands 48 hours after application (TO) and the right column shows the strands after 20 washing cycles performed 48 hours from application.

FIG. 8 shows images in accordance with Example 23 in which color gel 70A was applied to hair strands immediately after preparing the gel (TO), and at 5, 10, 20, 30 and 60 minutes thereafter. The bottom image shows the same strands after being stored for 48 hours at ambient conditions followed by 20 washing cycles performed 48 hours from application.

FIG. 9 shows images of hair strands in accordance with Example 25 after initial treatment with a silicone resin color gel+acrylate sealer and after 20 washing cycles.

FIG. 10 shows rub offs at 1, 24 and 48 hours in accordance with Example 25 in which various acrylate sealers were applied to hair strands after application of a silicone resin color gel.

### DETAILED DESCRIPTION OF THE INVENTION

The present invention is directed to silicone polyethyleneimine resins and compositions containing the resins for treatment or coloration of keratin fibers. The resins, methods of manufacturing the resins and application aspects of the invention are directed to embodiments of moisture-curable silicone polyethyleneimine resins obtained by reacting silylated polyethyleneimine with one or more hydrolyzable silanes having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H. At least one amino functional group of the silylated polyethyleneimine is covalently bonded with the first hydrolyzable silane group. The silylated polyethyleneimine may be reacted to the hydrolyzable silane already formed or it may be formed in situ by adding polyethyleneimine to the first hydrolyzable silane along with a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H. The mixture containing aminosilane is stirred at elevated temperature to react the components. The resulting silicone resins will cure at ambient conditions and can be combined with additional components, such as colorant, and applied to keratin fibers as a coating, sealant, adhesive or varnish.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

Hair and hair strands mean natural or synthetic keratin fibers. Hair, hair strands and keratin fibers are used interchangeably in this document. Natural keratin fibers include those from mammals and/or on mammals including human, primate, ruminant, camelid, equine, rodent and neovison including but not limited to cow, sheep, deer, goat, buffalo, lama, alpaca, camel, guanaco, vicuna, horse, antelope, moose, elk, rat, mouse, beaver, rabbit, mink, monkey, ape, and similar species. Natural keratin fibers may include hair, fur, or nails. Synthetic fibers include polyamides, polyacrylic and polyester fibers, especially polyamide fibers which are used for artificial hair implantation.

The term “in situ” is a Latin phase meaning in its original place. In the context of this invention, it means an activity such as a linking reaction or arrangement by covalent, coordinate, entanglement, ionic, hydrogen bonding, polar coupling, or electrostatic activity between two or more molecules that occurs in place, prior to application on keratinous materials.

“Alkyl” refers to a straight or branched hydrocarbon chain radical consisting solely of carbon and hydrogen atoms, unless otherwise specifically described as having additional heteroatoms or heterogroups. The alkyl group contains no unsaturation, having from one to twenty-two carbon atoms (e.g., C₁-C₂₄ alkyl). Whenever it appears herein, a numerical range such as “1 to 24 refers to each integer in the given range; e.g., “1 to 24 carbon atoms” means that the alkyl group may consist of 1 carbon atom, 2 carbon atoms, 3 carbon atoms, etc., up to and including 24 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

“Aryl” refers to a conjugated pi radical with six or ten ring atoms which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. The term includes monocyclic or monocyclic-ring polycyclic (i.e., rings which share adjacent pairs of ring atoms) groups.

“Silyl” refers to a —SiRₙ group as described herein.

“Vinyl” refers to the group —CH═CH₂.

“Alkoxy” refers to the group —O-alkyl, including from 1 to 24 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

“Aryloxy” refers to an —O-aryl group.

“Acetoxy” refers to —OC(═O)CH₃.

“Oximino” refers to RR′C═NOH methyl ethyl ketoximes and methyl isobutyl ketoxmines and may have either methyl ethyl ketoxime or methyl isobutyl ketoxmine groups and a single methyl, vinyl, or phenyl group, two methyl groups, or a methyl and vinyl group.

“Enoxy” refers to R—O—R′.

The term “halo” or “halogen” refers to fluorine (fluoro, —F), chlorine (chloro, —Cl), bromine (bromo, —Br), or iodine (iodo, —I).

“Amino” or “amine” refers to an —N(R^{a})₂ group, where each R^{a} is independently hydrogen or linear, branched, or cyclic alkyl of 1 to 6 carbons. When an —N(R^{a})₂ group has two R^{a} other than hydrogen they can be combined with the nitrogen atom to form a 4-, 5-, 6-, or 7-membered ring.

“Epoxyalkyl” refers to R¹HCOCH—R, where the oxygen is part of a cyclic ether composed of a three atom ring structure.

“(Meth)acryloxyalkyl” refers to CH₂═CR′COO—R—.

“Hydrolyzable silane” refers to a silane baring at least one hydrolyzable group Si—O—R.

The silicone resins are formed from at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H.

Examples of the such hydrolyzable silanes include triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof.

Exemplary alkoxysilanes can be chosen preferably from triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, and stearyltrimethoxysilane.

In certain embodiments, the at least one hydrolyzable silane comprises or is selected from the group consisting of triethoxymethylsilane, ethoxytrimethylsilane, diethoxydimethylsilane, tetraethoxysilane, vinyltriethoxysilane, hexyltriethoxysilane, n-octyltriethoxysilane, 1,8-bis(triethoxysilyl)octane, n-dodecyltriethoxysilane, stearyl triethoxysilane, and combinations thereof. Most preferably, the at least one hydrolyzable silane comprises or consists of methyltriethoxysilane (MTEOS).

The silicone resins are formed by reacting the hydrolyzable silanes having formula R¹ₙSiR²₄₋ₙ with polyethyleneimine and a linking organosilane or by adding silylated polyethyleneimine to the hydrolyzable silanes having formula R¹ₙSiR²₄₋ₙ.

### Polyethyleneimine

The polyethyleneimine used herein can include linear, branched, or a dendrimer polyethyleneimine. Preferably, the polyethyleneimine has a molecular weight ranging between 300 and 100,000 Daltons.

Exemplary selections include: a) Linear polyethyleneimine of the formula:

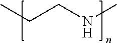

in which n is an integer representing the degree of polymerization, wherein n ranges from 5 to 25,000, alternatively from 11 to 2,500; and branched polyethyleneimine consisting of primary, secondary and tertiary amine groups of the formula:

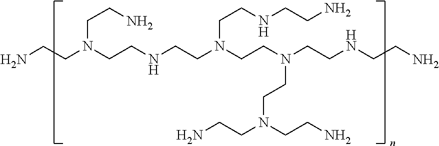

in which n is an integer representing the degree of polymerization, wherein n ranges from 2 to 4,000, alternatively from 5 to 500.

In particularly preferred embodiments, the polyethyleneimine is branched and has a molecular weight ranging from 300 to 1,200, more preferably 500 to 700.

The polyethyleneimine is silylated by combining with a hydrolyzable linking silane having formula R⁵ₙSiR⁶₄₋ₙ, wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H. In some embodiments, R⁵ is epoxyalkyl, (meth)acryloxyalkyl, or halogenalkyl, such as in 3-glycidoxypropyltriethoxysilane, or 3-chloropropyltriethoxysilane.

In some embodiments, the hydrolyzable linking silane having formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and mixtures thereof.

In some aspects, the hydrolyzable linking silane having formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-methacryloxypropyltrimethoxysilane, 3-acryloxypropyltrimethoxysilane, 3-acryloxypropyltrimethoxysilane, 3-acryloxypropyltrimethoxysilane, 3-chloropropyltrimethoxysilane, 3-chloropropyltriethoxysilane, and mixtures thereof.

In certain preferred embodiments, the hydrolyzable linking silane is 3-glycidoxypropyltrimethoxysilane.

In certain aspects, a molar ratio of the hydrolyzable silane having formula R⁵ₙSiR⁶₄₋ₙ to the polyethyleneimine is from about 1:100 to about 100:1, more preferably about 1:1 to 1:10, most preferably about 1:3 to 1:5.

As mentioned, the polyethyleneimine can be silylated prior to its addition to the at least one hydrolyzable silane of formula R¹ₙSiR²₄₋ₙ, or it can be silylated in situ in a process as described below.

### Catalyst

The formation of the silicone resin requires an acid or a base as a catalyst to link the at least one hydrolyzable silane with the silylated polyethyleneimine.

Examples of suitable catalysts include Lewis acids or bases or Bronsted acids or bases.

In some embodiments, the reaction will comprise at least one acid. This acid may be chosen from lactic acid, acetic acid, citric acid, tartaric acid, hydrochloric acid, sulfuric acid, and phosphoric acid. A preferable acid is hydrochloric acid. The hydrochloric acid (pure) may in particular be present in the reaction composition in a content of between 0.001% and 0.1% by weight and preferably in a content of between 0.01% and 0.05% by weight relative to the total weight of the composition.

In some embodiments, the reaction will comprise at least one base. The base may be an inorganic or organic compound. Illustrative of useful inorganic bases are ammonium hydroxide, alkali metal and alkaline earth metal hydroxides, carbonates and bicarbonates such as sodium hydroxide, lithium hydroxide, potassium hydroxide, calcium hydroxide, magnesium hydroxide, barium hydroxide sodium carbonate, sodium bicarbonate, calcium carbonate, calcium bicarbonate, and the like. The base is preferably potassium hydroxide. The potassium hydroxide (pure) may in particular be present in the reaction composition in a content of between 0.001% and 0.1% by weight and preferably in a content of between 0.01% and 0.05% by weight relative to the total weight of the composition.

In some embodiments, the acid or base is KOH, NaOH, LiOH, NH₄OH, NH₄CO₃, HCl, H₂SO₄, HNO₃, acidic clay, acidic ion exchange resin, or a mixture thereof. In certain embodiments, the acid or base is hydrochloric acid (HCl) or potassium hydroxide (KOH).

### Optional Hydrolyzable Aminosilane

The resins can be formed using an optional hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H.

Suitable aminosilanes having formula R³ₙSiR⁴₄₋ₙ include 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof.

In certain preferred embodiments, the aminosilane is 3-aminopropyltriethoxysilane, 3-aminopropyltrimethoxysilane, or 3-aminopropyltriethoxysilane. Among the alkoxysilane compounds mention may in particular be made of 3-aminopropyltriethoxysilane, 3-aminopropylmethyldiethoxysilaneand the oligomers formed from 3-aminopropyltriethoxysilane.

Most preferably, the hydrolyzable aminosilane is 3-aminopropyltriethoxysilane.

In certain aspects, a molar ratio of the hydrolyzable silane having formula R¹ₙSiR²₄₋ₙ to the aminosilane is from about 10:1 to about 1:10, preferably about 10:1 to about 1:1, more preferably about 7:1 to about 3:1, most preferably about 3:1 to about 4:1.

### Solvent

The reaction to form the resin may contain a solvent, such as alcohol, water, aliphatic hydrocarbons, aromatic hydrocarbons, ether, tetrahydrofuran, ethyl acetate, methyl ethyl ketone, or a mixture thereof.

### Process of Preparing Silicone Resins

Silylated polyethyleneimine can be prepared by reacting polyethyleneimine and a linking silane. The reaction will typically be carried out at a temperature of about 50-200° C., more preferably about 70-120° C., most preferably about 80-95° and will typically be allowed to stir for 5 minutes to 12 hours, more preferably 10 minutes to 6 hours, most preferably about 1 to about 3 hours.

Exemplary silylated polyethylenimines were made with the following ingredients and reaction conditions

| Linking Silicone | BPEI | | |
|---|---|---|---|
| R⁵ₙSiR⁶₄₋ₙ | (Mw) | Ratio | Reaction Conditions |
| GPTMS | 600 | 1:4 | 90° C, 3 hours |
| GPTMS | 600 | 1.5:1 | 90° C, 3 hours |
| GPTMS | 600 | 4:1 | 90° C, 3 hours |
| ISOCYANPTES | 600 | 1:4 | 90° C, 3 hours |
| 3CIPTEOS | 600 | 1:4 | 90° C, 3 hours |
| 3CIPTEOS | 600 | 1.5:1 | 90° C, 3 hours |
| MAPTEOS | 600 | 1:4 | 90° C, 3 hours |
| MAPTEOS | 600 | 1.5:1 | 90° C, 3 hours |

MAPTEOS = 3-methacryloxypropyltriethoxysilane
GPTMS = 3-glycidoxypropyltrimethoxysilane
3CIPTEOS = 3-cloropropyltriethoxysilane
ISOCYANPTES = 3-isocyanatopropyltriethoxysilane

The silylated polyethyleneimine can also be formed in situ. The reaction will typically require a step of heating a mixture of the various reaction components above room temperature, such as to about 50-200° C., more preferably about 70-120° C., most preferably about 80-95° C. for 5 minutes to 12 hours, more preferably 10 minutes to 6 hours, most preferably about 1 to about 3 hours.

In one exemplary process for preparing a moisture-curable silicone resin, at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H is stirred with silylated polyethyleneimine at a temperature of from 0° C. to 100° C., more preferably about 10-50° C., most preferably about 20-40° C., for 0.1 to 12 hours, such as 1 minute to 6 hours, more preferably 5 minutes to 3 hours, more preferably 10 minutes to 1.5 hours, most preferably about 20 to 45 minutes. Then, a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H is added and the reaction mixture is heated to 50-200° C., more preferably about 70-120° C., most preferably about 80-95° C. for 5 minutes to 12 hours, more preferably 10 minutes to 6 hours, most preferably about 1 to about 3 hours.

In certain embodiments, it may not be necessary to add a hydrolyzable aminosilane to form the moisture-curable silicone resin. That is, a reaction to form the silicone resins described herein will consist of a hydrolyzable silane having formula R¹ₙSiR²₄₋ₙ, polyethyleneimine silylated with a hydrolyzable silane having formula R⁵ₙSiR⁶₄₋ₙ, solvent, and acid or base. In other embodiments, a reaction to form the silicone resins described herein will consist of a hydrolyzable silane having formula R¹ₙSiR²₄₋ₙ, and polyethyleneimine silylated with a hydrolyzable silane having formula R⁵ₙSiR⁶₄₋ₙ, hydrolyzable aminosilane having formula R³ₙSiR⁴₄₋ₙ, solvent, and acid or base. The process will consist essentially of mixing at least one hydrolyzable silane having formula R¹ₙSiR²₄₋ₙ with silylated polyethyleneimine, water, acid or base and heating the mixture.

Another process for preparing a moisture-curable silicone resin comprises
(i) Mixing e.g., stirring, at a temperature of from 0° C. to 100° C., preferably at about 10-50° C., most preferably about 20-40° C. for 0.1 to 12 hours, preferably 1 minute to 6 hours, more preferably 5 minutes to 3 hours, more preferably 10 minutes to 1.5 hours, most preferably about 20 to 45 minutes
a. water,
b. an acid or a base,
c. at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H; and
d. a hydrolyzable silane having formula R⁵ₙSiR⁶₄₋ₙ, wherein R⁵ is epoxyalkyl, (meth)acryloxyalkyl, acryloxyalkyl, halogenalkyl, isocyantoalkyl, or a combination thereof; R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof; n=1, 2, or 3; R is alkyl or H;
(ii) adding polyethyleneimine to the mixture of step (i) and stirring 0.1 to 12 hours, such as 5 minutes to 3 hours, more preferably 10 minutes to 1.5 hours, most preferably about 20 to 40 minutes at a temperature of 0° C. to 100° C., more preferably at about 10-50° C., most preferably about 20-40° C.;
(iii) adding to the reaction of step (ii) a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H; and
(iv) heating the reaction of step (iii) for at least 30 minutes. The reaction is typically heated to 50-200° C., more preferably about 70-120° C., most preferably about 80-95° C. for 5 minutes to 12 hours, more preferably 10 minutes to 6 hours, most preferably about 1 to about 3 hours.

Alternatively, the d. linking silane having formula R⁵ₙSiR⁶₄₋ₙ can be added together with or after the polyethyleneimine is added. The reaction to form the resin may contain additional solvent, such as alcohol, aliphatic hydrocarbons, aromatic hydrocarbons, ether, tetrahydrofuran, ethyl acetate, methyl ethyl ketone, or a mixture thereof.

A further process involves mixing (e.g., stirring) at a temperature of from 0° C. to 100° C., more preferably about 10-50° C., most preferably about 20-40° C., for 0.1 to 12 hours, an acid or a base, and at least one hydrolyzable silane having formula R¹ₙSiR²₄₋ₙ, wherein R¹ is alkyl, aryl, fluoroalkyl, trialkylsilyl, triarylsilyl, vinyl, or a combination thereof; R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof; n=0, 1, 2, or 3; R is alkyl or H. To the reaction is added silylated polyethyleneimine and a hydrolyzable aminosilane having formula R³ₙSiR⁴₄₋ₙ, wherein R³ is aminoalkyl, aminoaryl, or a combination thereof; R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof; n=1, 2, or 3; R is alkyl or H. In some embodiments, after adding aminosilane, the reaction is mixed at a temperature of about 10-50° C., more preferably about 20-40° C. In certain embodiments, the reaction is mixed at about 60° C. to about 200° C., or about 70° C. to about 120° C., or about 80° C. to about 95° C. In certain embodiments, mixing occurs for 30 minutes to 6 hours, or about 1 hour to about 3 hours.

The reaction to form the resin may contain a solvent, such as alcohol, water, aliphatic hydrocarbons,-aromatic hydrocarbons, ether, tetrahydrofuran, ethyl acetate, methyl ethyl ketone, or a mixture thereof.

In certain embodiments, a reaction to form the silicone resins described herein will consist of a hydrolyzable silane having formula R¹ₙSiR²₄₋ₙ, water, an acid or a base, polyethyleneimine, a hydrolyzable silane having formula R⁵ₙSiR⁶₄₋ₙ, and hydrolyzable aminosilane having formula R³ₙSiR⁴₄₋ₙ. In other embodiments, a reaction to form the silicone resins described herein will consist of two hydrolyzable silanes having formula R¹ₙSiR²₄₋ₙ, water, an acid or a base, polyethyleneimine, a hydrolyzable silane having formula R⁵ₙSiR⁶₄₋ₙ, and hydrolyzable aminosilane having formula R³ₙSiR⁴₄₋ₙ. In yet other embodiments, a reaction to form the silicone resins described herein will consist of three hydrolyzable silanes having formula R¹ₙSiR²₄₋ₙ, water, an acid or a base, polyethyleneimine, a hydrolyzable silane having formula R⁵ₙSiR⁶₄₋ₙ, and hydrolyzable aminosilane having formula R³ₙSiR⁴₄₋ₙ.

In certain embodiments, a reaction to form the silicone resins described herein will consist of a hydrolyzable silane having formula R¹ₙSiR²₄₋ₙ, water, an acid or a base, polyethyleneimine, and a hydrolyzable silane having formula R⁵ₙSiR⁶₄₋ₙ. In other embodiments, a reaction to form the silicone resins described herein will consist of two hydrolyzable silanes having formula R¹ₙSiR²₄₋ₙ, water, an acid or a base, polyethyleneimine, and a hydrolyzable silane having formula R⁵ₙSiR⁶₄₋ₙ. In yet other embodiments, a reaction to form the silicone resins described herein will consist of three hydrolyzable silanes having formula R¹ₙSiR²₄₋ₙ, water, an acid or a base, polyethyleneimine, and a hydrolyzable silane having formula R⁵ₙSiR⁶₄₋ₙ.

Exemplary reaction components of silicone resins in which there is in situ formation of silylated polyethyleneimine are shown in the table below. The components consist of one to three hydrolyzable silanes having formula R¹ₙSiR²₄₋ₙ, an acid or a base, branched polyethyleneimine, a polyethyleneimine linking silane having formula R⁵ₙSiR⁶₄₋ₙ, water, and preferably, an aminosilane having formula R³ₙSiR⁴₄₋ₙ. The amount of water ranges from 1-30%, more preferably 1-20%. Typically, if a reaction is based catalyzed, a higher weight percentage of water can be used without leading to gelation. In certain embodiments, water is around 7% by weight of the reaction components.

| Hydrolyzable | Acid or | PEI Linking | | Optional |
|---|---|---|---|---|
| Silane | Base | Silicone | BPEI | Aminosilane |
| R¹ₙSiR²₄₋ₙ | Catalyst | R⁵ₙSiR⁶₄₋ₙ | (Mw) | R³ₙSiR⁴₄₋ₙ |
| MTEOS (T) | HCl | MAPTEOS | 600 | AMEO |
| MTEOS (T) | HCl | GPTMS | 600 | AMEO |
| MTEOS (T) | HCl | 3CIPTEOS | 600 | AMEO |
| MTEOS (T) | HCl | GPTMS | 600 | AMEO |
| MTEOS (T) + | HCl | GPTMS | 600 | AMEO |
| n-Octyltri- | | | | |
| ethoxysilane | | | | |
| MTEOS (T) | KOH | MAPTEOS | 600 | AMEO |
| MTEOS (T) | HCl | GPTMS | 600 | AMEO |
| MTEOS (T) + | HCl | GPTMS | 600 | AMEO |
| DEODMS (D) + | | | | |
| TEOS (Q) | | | | |
| MTEOS (T) | HCl | MAPTEOS | 600 | AMEO |
| MTEOS (T) | HCl | GPTMS | 300 | AMEO |
| MTEOS (T) | HCl | GPTMS | 1200 | AMEO |
| MTEOS (T) | HCl | ISOCYANPTES | 600 | AMEO |
| MTEOS (T) | HCl | MAPTEOS | 600 | — |
| MTEOS (T) + 1,8- | HCl | GPTMS | 600 | AMEO |
| bis(Triethoxy- | | | | |
| silyl)octane | | | | |

MTEOS = Methyltriethoxysilane
DEODMSdiethoxydimethylsilane
TEOS = triethoxytrimethylsilane
MAPTEOS = methacryloxypropyltriethoxysilane
GPTMS = 3-glycidoxypropyltrimethoxysilane
3CIPTEOS = 3-cloropropyltriethoxysilane
ISOCYANPTES = 3-isocyanatopropyltriethoxysilane
BPEI = branched polyethyleneimine
AMEO = 3-aminopropyltriethoxysilane

The nomenclature of silicone resins is known under the name “MDTQ,” the resin being described as a function of the various siloxane monomer units it comprises, each of the letters M, D, T and Q characterizing a type of unit. The letter M represents the monofunctional unit of formula (CH₃)₃SiO_{1/2}, the silicon atom being connected to only one oxygen atom in the polymer comprising this unit. The letter D means a difunctional unit (CH₃)₂SiO_{2/2} in which the silicon atom is connected to two oxygen atoms. The letter T represents a trifunctional unit of formula (CH₃)SiO_{3/2}. In the units M, D and T defined previously, at least one of the methyl groups may be substituted with a group R other than a methyl group, such as a hydrocarbon-based radical (for example alkyl) containing from 2 to 10 carbon atoms or a phenyl group, or alternatively a hydroxyl group. Finally, the letter Q means a tetrafunctional unit SiO_{4/2} in which the silicon atom is bonded to four hydrogen atoms, which are themselves bonded to the rest of the polymer.

### Cosmetic Compositions

The silicone resins can be used in or as cosmetic compositions. The resins and compositions containing them can be applied as a coating, sealant, adhesive, or varnish, typically on keratinous materials.

The resins of the present invention may be used to prepare simple aqueous compositions for application to the hair, such as an aqueous “leave on” composition or an aqueous “rinse off” composition. The compositions may further be useful as a nail varnish or varnish base.

The silicone resins may be combined with solvents, carriers, coloring agents, plasticizers, vitamins, fragrances, trace elements, softeners, plasticizers, coalescers, preserving agents, stabilizers, co-resins, anti-foams, spreading agents and the like to form suitable cosmetic compositions.

The amounts of these various ingredients are those conventionally used in this field, for example from 0.01% to 20% by weight and especially from 0.02% to 10% by weight relative to the total weight of the composition in accordance with the invention.

Suitable coloring agents include pigments, direct dyes, or a mixture thereof.

In some embodiments, the coloring agent includes compound from the group of organic pigments, such as carmine, quinacridone, phthalocyanine, sorghum, blue pigments having the color index numbers CI 42090, CI 69800, CI 69825, CI 73000, CI 74100, CI 74160, yellow pigments having the color index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and CI 75470. In one particular embodiment, the coloring agent comprises CI 15880.

In some embodiments, the coloring agent includes at least one direct dye which is selected from the group of anionic, cationic, and nonionic direct dyes. Anionic direct dye may be selected from the Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D & C Yellow 8, D & C Green 5, D & C Orange 10, D & C Orange 11, D & C Red 21, D & C Red 27, D & C Red 33, D & C Violet 2 and D & C Brown 1.

The coloring agent(s) will typically comprise from 0.1% to 40% by weight, for example ranging from 0.1% to 30% by weight, such as ranging from 0.5% to 20% by weight, such as ranging from 1% to 20% by weight and for instance ranging from 1% to 15% by weight, relative to the total weight of the composition.

A composition according to the invention comprises a physiologically acceptable medium. The term “physiologically acceptable medium” is intended to mean a non-toxic medium that may be applied to human keratin materials and that has a pleasant appearance, odor and feel.

A cosmetic composition according to the invention may also comprise a film-forming polymer, such as polyethylene glycol. The term “film-forming” polymer means a polymer that is capable, by itself or in the presence of an auxiliary film-forming agent, of forming a macroscopically continuous film on a support, for example on keratin materials, and for instance a cohesive film. In certain embodiments, a water-soluble film-forming polymer may be preferred.

The film-forming polymer may be present in the compositions according to the disclosure in an amount ranging from 0.1% to 40% by weight, for example ranging from 0.1% to 30% by weight, such as ranging from 0.5% to 20% by weight, such as ranging from 1% to 20% by weight and for instance ranging from 1% to 15% by weight, relative to the total weight of the composition.

### Coating Kit

When cosmetic compositions according to the present invention contain a mixture of moisture-curable silicone resin and coloring agent, they can also be in the form of a kit for coating keratin materials comprising at least two different compositions packaged separately. Said kit may comprise the product resulting from the combination.

A kit for coloring a keratin fiber can comprise a first composition comprising the moisture-curable silicone resin and a second composition comprising the at least one coloring agent selected from pigments, direct dyes, or a mixture thereof. The content of the first and the second compositions are mixed into a ready-to-use silicone resin color composition prior to application to the keratin fiber.

In some embodiments, the at least one coloring agent comprises about 0.5% to about 10% by weight, preferably about 1-5% by weight of the first composition. The ready-to-use silicone resin color composition can comprise about 5% to about 20%, preferably about 10% by weight of the silicone resin and about 0.5% to about 2% by weight of coloring agent.

The two different compositions may be combined just before or during application, preferably before application.

Said first and/or second different compositions of the kit according to the invention may also comprise at least one additional compound as defined previously, such as a film forming polymer, a surfactant, an acid or a base, water and/or a solvent.

The kit can also comprise a sealer composition. The sealer can be a UV curable sealer comprising at least one photoinitiatior and at least one acrylate monomer or oligomer. The photoinitiator can be ethyl (2,4,6-trimethylbenzoyl) phenylphosphinate, diphenyl(2,4,6-trimethylbenzoyl)phosphine oxide, phenylbis(2,4,6-trimethylbenzoyl)phosphine oxide, or mixture thereof. The acrylate monomer or oligomer may be a multifunctional acrylate having a molecular weight between 200 and 10,000 Daltons.

Each composition of the kit may be packaged separately in the same packaging article.

Each composition may also be packaged in a separate compartment within the same packaging article, the mixing of the at least two compositions taking place at the end(s) of the packaging article during the delivery of the composition. Preferably, the sealer is not combined with the first or second composition before or after packaging. The sealer is optionally applied after the silicone resin and/or color as discussed below. The packaging article(s) may be watertight and/or airtight. Alternatively, each composition may be packaged in a different packaging article.

### Application

Color treatment compositions as disclosed herein can be applied to hair a manner known in the art. For instance, in certain embodiments, a silicone resin color gel can be applied to hair strands by the methods disclosed in Morel O. J. X., Christie R. M., Current Trends in the Chemistry of Hair Dyeing Chem Rev., 2011, 111, 2537-256.

Preferably, the application is performed on dry fibers. The coated fibers are exposed to ambient conditions for 0.1 to 48 hours. According to one particular embodiment, said coating process is performed with application of heat. The composition may also be heated using a means specifically dedicated to heating, for instance a means for propelling hot air such as a hair dryer or a drying device, for instance a heating applicator.

Subsequent to application of a silicone polyethyleneimine resin as disclosed herein, an acrylate sealer or cellulose composition may optionally be applied to the hair strands. In such embodiments, it is preferable to partially cure the silicone polyethyleneimine resin. For instance, if a resin would typically fully cure in 30 minutes at ambient conditions, the resin (and any colorant) can be applied to hair and cured for 15 minutes followed by application of an acrylate sealer. Various acrylate sealers are commercially available and known in the art and can be applied as directed.

In one embodiment, an acrylate sealer can be applied, e.g., 1-60 minutes after application of a silicone polyethyleneimine resin described herein. The sealer can sit on the hair for a directed amount of time depending upon the sealer composition, such as 1-15 minutes. After application of the sealer, the hair can be rinsed with water and exposed to UV light. Thereafter, the UV-treated strands can be rinsed, conditioned and blow dried. In some embodiments, after the acrylate sealer is applied, it is cured with application of heat.

Various acrylate sealers were found not to impact longevity of color but to help reduce the wait time needed before washing colored hair. Use of acrylate sealer may also improve rub off from hair of a silicone polyethyleneimine color formulation.

Without being bound by theory, when the silicone resin containing compositions are applied to material, ambient humidity will hydrolyze the unreacted alkoxysilane groups and then condense to form a long lasting coating layer. For example, ambient humidity ranging from 10% RH to 85% RH has been found sufficient to cure the resins. Surprisingly, however, initial exposure to water or wet hair was not found to have superior performance and may rinse off the resin before it can sufficiently cure and coat the hair. In some embodiments, it may be critical that the ambient humidity is below 85% RH, or below 60% RH, or below 50% RH, or about 15% RH to about 45% RH for at least 10 minutes after application of the moisture-curable resins.

### EXAMPLES

In the following examples, silane monomers were predominantly acquired from GELEST, branched polyethyleneimines were acquired either from Beantown Chemical, Nippon Shockubay or Sigma Aldrich, Acrylates were acquired from Sartomer, pigments were acquired from Sensient, all other chemicals were acquired from Sigma Aldrich or BASF and used without further purification. Hair samples were acquired from Kerling.

IR spectra were acquired on Perkin-Elmer Spectrum 65, with Software Perkin Elmer Spectrum IR version 10.6.2.

Humidity Chamber MODEL: BLT-433 was used.

Room temperature was about 20-25° C.

### Example 1—Synthesis of Silicone Resin

Methyltriethoxysilane (MTEOS) 23.89 g (134 mmol), 3-methacryloxypropyltriethoxysilane (MAPTEOS) 0.129 g (0.445 mmol), water 1.44 g, HCl 0.5M 1.33 g, were added to a 100 mL 3 neck round bottom flask equipped with a condenser under magnetic stirring, and allowed to react at room temperature for 30 min. BPEI 600 1.068 g (2 mmol) was added, and the mixture was allowed to react for 2 h at room temperature, then 3-aminopropyltriethoxysilane (AMEO) 7.49 g (33.8 mmol) was added and the mixture reaction was allowed to react for 30 min at room temperature, and then at 90° C. for 1 h. The resulting product was either packed out in a glass jar, or further concentrated by removal of the residual volatiles under reduced pressure at 80° C. for 1 h.

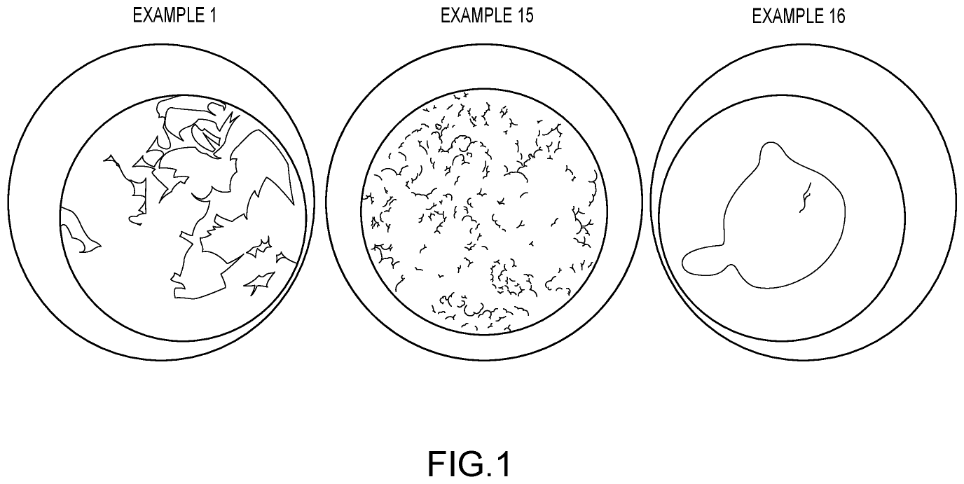

2 mL of the resin were placed in an aluminum pan and cured at ambient conditions producing a brittle film. FIG. 1 shows an image of the resulting resin after curing at ambient conditions alongside Examples 15 and 16 (from left to right). Changing the BPEI concentration and the presence or absence of AMEO in association with the BPEI resin enables tuning of the mechanical properties of the formed film towards performance and application.

### Example 2—Synthesis of Silicone Resin

Methyltriethoxysilane (MTEOS) 23.89 g (134 mmol), water 1.44 g, HCl 0.5M 1.33 g, were added to a 100 mL 3 neck round bottom flask equipped with a condenser under magnetic stirring, and allowed to react at room temperature for 30 min. BPEI 600 1.068 g (2 mmol) was added and subsequently 3-glycidoxypropyltrimethoxysilane (GPTMS) 0.105 g (0.445 mmol) were added and the reaction was allowed to react for 2 h at room temperature, then 3-aminopropyltriethoxysilane (AMEO) 7.49 g (33.8 mmol) was added and the reaction was allowed to react for 30 min at room temperature, and then at 90° C. for 1 h. The resulting product was either packed out in a glass jar, or further concentrated by removal of the residual volatiles under reduced pressure at 80° C. for 1 h.

6 mL of the resin was placed in an aluminum pan. Topographic differences were observed as compared to resin made without BPEI; specifically, the BPEI free resin presents fracture points and bending as a consequence of shrinkage generated during curing following the evaporation of solvent. The shrinkage was mitigated in the BPEI containing resin. The Si-BPEI resin also appeared to present a higher wrinkle density compared to the BPEI free control, potentially generating interesting diffracting light patterns that can impact color performance.

### Example 3—Synthesis of Silicone Resin

Methyltriethoxysilane (MTEOS) 47.784 g (268 mmol), 3-cloropropyltriethoxysilane (3CIPTEOS) 0.241 g (1 mmol), water 2.883 g, HCl 0.5M 2.66 g, were added to a 250 mL 3 neck round bottom flask equipped with a condenser under magnetic stirring, and allowed to react at room temperature for 30 min, then 2.136 g (4 mmol) of BPEI 600 were added and the reaction was allowed to react for 2 h at room temperature, then 3-aminopropyltriethoxysilane (AMEO) 14.83 g (67 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 4—Synthesis of Silylated BPEI

BPEI 600 10 g (16.667 mmol) was added to a 3 neck round bottom flask under magnetic stirring and subsequently 3-glycidoxypropyltrimethoxysilane (GPTMS) 1.0 g (4.153 mmol) was added and allowed to react at 90° C. for 3h.

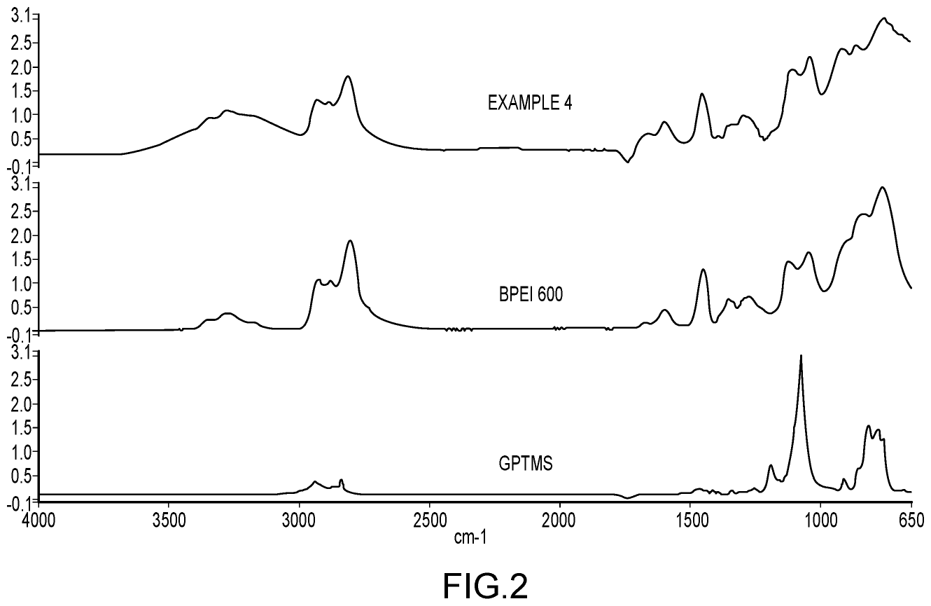

FIG. 2 shows IR spectrum of resulting product against the starting materials. There is a decrease in the peak at 2807 from the BPEI 600 spectrum to product of Example 4 and a widening of the bands between 3000 and 3500 and the appearance/increase of the shoulder at 1657.34 indicating complete reaction of GPTMS. The result was confirmed by GC, where GPTMS was below detection limit (<20 ppm).

### Example 5—Synthesis of Silicone Resin

Methyltriethoxysilane (MTEOS) 48.498 g (272 mmol) and 6.4 g of Example 4 were added to a 250 mL 3 neck round bottom flask equipped with a condenser under magnetic stirring, and allowed to react at room temperature for 2 h, then 3-aminopropyltriethoxysilane (AMEO) 14.97 g (67.6 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1 h.

### Example 6—Synthesis of Silicone Resin

Methyltriethoxysilane (MTEOS) 42.970 g (241 mmol), n-octyltrithoxysilane 7.410 (27 mmol), water 3.244 g, HCl 0.5M 2.66 g, were added to a 250 mL 3 neck round bottom flask equipped with a condenser under magnetic stirring, and allowed to react at room temperature for 30 min. BPEI 600 2.136 g (4 mmol) and subsequently 3-glycidoxypropyltrimethoxysilane (GPTMS) 0.210 g (1 mmol) were added and the reaction was allowed to react for 2 h at room temperature, then 3-aminopropyltriethoxysilane (AMEO) 14.83 g (67 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 7—Synthesis of Silicone Resin

Methyltriethoxysilane (MTEOS) 23.89 g (134 mmol), 3-methacryloxypropyl triethoxysilane (MAPTEOS) 0.129 g (0.445 mmol), water 1.44 g, KOH 0.5M 1.33 g, were added to a 100 mL 3 neck round bottom flask equipped with a condenser under magnetic stirring, and allowed to react at room temperature for 30 min. BPEI 600 1.068 g (2 mmol) of BPEI 600 was added and the reaction was allowed to react for 2 h at room temperature, then 3-aminopropyltriethoxysilane (AMEO) 7.49 g (33.8 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 8—Synthesis of Silicone Resin

Methyltriethoxysilane (MTEOS) 23.89 g (134 mmol), 3-methacryloxypropyl triethoxysilane (MAPTEOS) 0.129 g (0.445 mmol), water 1.44 g, KOH 0.5M 1.33 g, were added to a 100 mL 3 neck round bottom flask equipped with a condenser under magnetic stirring, and allowed to react at room temperature for 30 min. BPEI 600 1.068 g (2 mmol) of BPEI 600 was added and the reaction was allowed to react for 2 h at room temperature, then 3-aminopropyltriethoxysilane (AMEO) 7.49 g (33.8 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 9—Synthesis of Silicone Resin

Methyltriethoxysilane (MTEOS) 43.030 g (241 mmol), diethoxydimethylsilane (DEODMS) 0.997 g (6.72 mmol), triethoxytrimethylsilane (TEOS) 3.501 g (17 mmol), water 2.883 g, HCl 0.5M 2.66 g, were added to a 250 mL 3 neck round bottom flask equipped with a condenser under magnetic stirring, and allowed to react at room temperature for 30 min. BPEI 600 2.136 g (4 mmol) and subsequently 3-glycidoxypropyltrimethoxysilane (GPTMS) 0.210 (1 mmol) were added and the reaction was allowed to react for 2 h at room temperature then 3-aminopropyltriethoxysilane (AMEO) 14.97 g (67.6 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 10—Synthesis of Silicone Resin

Methyltriethoxysilane (MTEOS) 23.89 g (134 mmol), 3-methacryloxypropyl triethoxysilane (MAPTEOS) 0.129 g (0.445 mmol), water 1.44 g, HCl 0.5M 1.33 g, were added to a 100 mL 3 neck round bottom flask equipped with a condenser under magnetic stirring, and allowed to react at room temperature for 30 min. BPEI 600 1.068 g (2 mmol) was added and the reaction was heated to 90° and allowed to react for 2 h, then 3-aminopropyltriethoxysilane (AMEO) 7.49 g (33.8 mmol) was added and the reaction was allowed to react for an additional 1 h. The resulting product was either packed out in a glass jar, or further concentrated by removal of the residual volatiles under reduced pressure at 80° C. for 1 h.

### Example 11—Synthesis of Silicone Resin

Methyltriethoxysilane (MTEOS) 23.89 g (134 mmol), water 1.44 g, HCl 0.5M 1.33 g, were added to a 100 mL 3 neck round bottom flask equipped with a condenser under magnetic stirring, and allowed to react at room temperature for 30 min. BPEI 300 0.6 g (2 mmol) and subsequently 3-glycidoxypropyltrimethoxysilane (GPTMS) 0.105 g (0.445 mmol) were added and the reaction was allowed to react for 2 h at room temperature, then 3-aminopropyltriethoxysilane (AMEO) 7.49 g (33.8 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 12—Synthesis of Silicone Resin

Methyltriethoxysilane (MTEOS) 23.89 g (134 mmol), water 1.44 g, HCl 0.5M 1.33 g, were added to a 100 mL 3 neck round bottom flask equipped with a condenser under magnetic stirring, and allowed to react at room temperature for 30 min, BPEI 1200 2.4 g (2 mmol) of and subsequently 3-glycidoxypropyltrimethoxysilane (GPTMS) 0.105 g (0.445 mmol) were added and the reaction was allowed to react for 2 h at room temperature, then 3-aminopropyltriethoxysilane (AMEO) 7.49 g (33.8 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1h.

### Example 13—Synthesis of Silylated BPEI—Free of AMEO

BPEI 600 10 g (16.667 mmol) was added to a 3 neck round bottom flask under magnetic stirring and subsequently 3-isocyanatopropyl triethoxysilane (ISOCYANPTES) 0.85 g (4.153 mmol) was added and allowed to react at 90° for 3h.

### Example 14—Synthesis of Silicone Resin

Methyltriethoxysilane (MTEOS) 48.498 g (272 mmol) and 2.8 g of example 15, were added to a 250 mL 3 neck round bottom flask equipped with a condenser under magnetic stirring and allowed to react at room temperature for 2 h at room temperature, then 3-aminopropyltriethoxysilane (AMEO) 7.49 g (33.8 mmol) was added and the reaction was allowed to react for 30 min at room temperature and then at 90° C. for 1 h. The resulting product was either packed out in a glass jar, or further concentrated by removal of the residual volatiles under reduced pressure at 80° C. for 1 h.

### Example 15—Synthesis of Silicone Resin—Free of AMEO

Methyltriethoxysilane (MTEOS) 95.568 (536 mmol), 3-methacryloxypropyl triethoxysilane (MAPTEOS) 0.517 g (2 mmol), water 5.76 g, HCl 0.5M 5.32 g, were added to a 250 mL 3 neck round bottom flask equipped with a condenser under magnetic stirring and allowed to react at room temperature for 30 min. BPEI 600 4.27 g (7 mmol) was added, and the mixture was allowed to react for 2 h at room temperature, then heated at 90° C. for 1 h. The resulting product was either packed out in a glass jar, or further concentrated by removal of the residual volatiles under reduced pressure at 80° C. for 1h.

2 mL of the resin was placed in an aluminum pan and cured at ambient conditions producing a rubbery film. FIG. 1 shows an image of the resulting resin after curing at ambient conditions.

### Example 16—Synthesis of Silicone Resin—Free of AMEO

Methyltriethoxysilane (MTEOS) 10.475 (58.75 mmol), 3-methacryloxypropyltriethoxysilane (MAPTEOS) 1.208 g (4 mmol), water 0.681 g, HCl 0.5M 0.63 g, were added to a 100 mL 3 neck round bottom flask equipped with a condenser under magnetic stirring and allowed to react at room temperature for 30 min. BPEI 600 9.96 g (17 mmol) was added, and the mixture was allowed to react for 2 h at room temperature, and then heated at 90° C. for 1 h. The resulting product was either packed out in a glass jar, or further concentrated by removal of the residual volatiles under reduced pressure at 80° C. for 1 h.

2 mL of the resin was placed in an aluminum pan. FIG. 1 shows an image of the resulting resin after curing at ambient conditions.

### Example 17—Synthesis of Silylated BPEI—Free of AMEO

BPEI 600 10 g (16.667 mmol) was added to a 3 neck round bottom flask under magnetic stirring and subsequently 3-cloropropyltriethoxysilane (3CIPTEOS) 1.0 g (4.153 mmol) was added and allowed to react at 90° for 3h.

### Example 18—Synthesis of Silylated BPEI—Free of AMEO

BPEI 600 10 g (16.667 mmol) was added to a 3 neck round bottom flask under magnetic stirring and subsequently 3-metacriloyloxypropiltriethoxysilane (MAPTEOS) 1.2 g (4.153 mmol) was added and allowed to react at 90° C. for 3h.

### Example 19—Comparative Synthesis—Free of BPEI

Methyltriethoxysilane (MTEOS) 71.67 g (402 mmol), water 4.325 g, HCl 0.5M 4.0 g, were added to a 250 mL 3 neck round bottom flask equipped with a condenser under magnetic stirring, and allowed to react at room temperature for 30 min. Then 3-aminopropyltriethoxysilane (AMEO) 23.64 g (107 mmol) was added and the reaction was allowed to react for 30 min at room temperature, and then at 90° C. for 1 h. The resulting product was either packed out in a glass jar, or further concentrated by removal of the residual volatiles under reduced pressure at 80° C. for 1 h.

### Example 20—Viscosity of Silylated BPEI

The viscosity of BPEI 600 and the resins of Examples 4, 17, and 18 was measured using a Brookfield viscosimeter, spindle 2, at 25° C. and 50 rpm.

| SAMPLE | VISCOSITY |
|---|---|
| BPEI 600 | 2295 cP |
| Example 4 | 4222 cP |
| Example 17 | 5340 cP |
| Example 18 | 2820 cP |

### Example 21—Preparation of Silicone Resin Color Gels

In a 500 mL beaker 400 g of PEG400 were added to 100 g of PEG6000 under agitation with an IKA RW16 mixer at 80° C. and the mixture was then allowed to cool under stirring. The gel color was prepared by adding to 80.5 g of the peg gel 8.9 g of a solution of 10% UNIPURE Red LC3079, 10% PLANTACARE 2000 and 80% water. The mixture was stirred with the IKA RW16 mixer until color appeared mixed homogeneously. To 9 g gel color 10% by weight of a silicone resin was added, the mixture was stirred and immediately applied onto the hair swatch as set forth in Example 22.

| Sample | Example | Comment |
|---|---|---|
| 37A | 19 | MTEOS + AMEO (No BPEI) |
| 38/38A | 1 | MTEOS + AMEO + MAPTEOS + BPEI 600 |
| 70A | 2 | MTEOS + AMEO + GPTMS + BPEI 600 |

### Example 22—Hair Treatment with Color Gels

Hair samples were acquired from Kerling and a sebum-mimicking preparation was applied, followed by application of a silicone resin color gel. Rub Offs to determine fastness of the silicone resin color gels were taken at 1 h; 24 h and 48h. After 48 hours, the longevity of the color was determined after 20 washes.

### General Sebum Preparation and Application Procedure:

Artificial sebum was prepared adding 80 g of squalane to 20 g C₁₀-20 trigliceride into a 150 mL beaker under magnetic stirring at 80° C., the mixture was left to cool down under magnetic stirring. [00237]200 mg of artificial sebum were added on Al Pan, blonde hair swatch of average weight around 500 mg is added to the Al Pan and sebum is layered on the hair swatch from root to tips. Then swatch is patted with absorbing paper towel, rinsed under running water, and blow dried.

### Application of Silicone Resin Color Gel

Application consisted of adding to Al pan 300 to 450 mg of Color Gel of Example 21 and immersing a hair swatch, and layering color on swatch from root to tip.

### Rub Off Procedure

Rub Offs were taken at 1 h; 24 h and 48h by placing the hair swatch between a folded 8×8 cm square of WypAll© from Kimberly-Clark professional and placing a 500 g weight on top of it, then sliding the swatch out of the paper pocket. The hair swatches were stored at 23° C. either low humidity (16% RH) or high humidity (45% RH).

### General Longevity Test Protocols

Swatches underwent 20 washing cycles at 48h from color treatment. One washing cycle consists of 6 steps: 1) wet swatch under running water for a few seconds, 2) apply shampoo 3) rinse 4) condition 5) rinse and 6) blow dry.

### Results

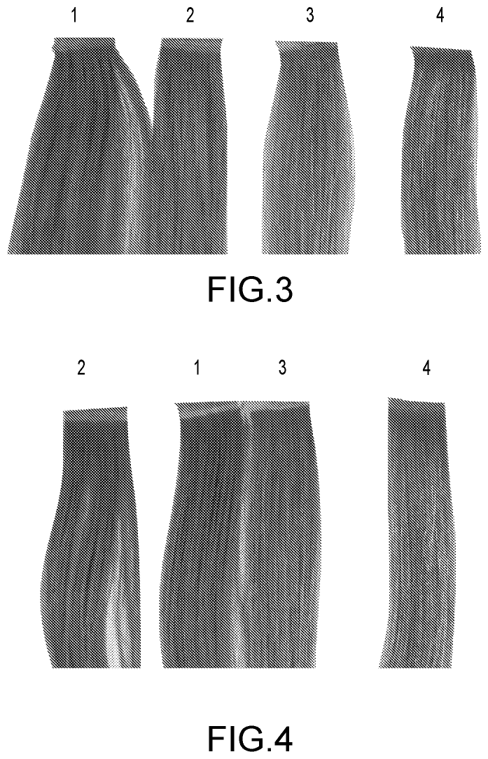

FIG. 3 shows a comparison of color gel 37A (no BPEI) applied to hair that has not been treated with sebum (1 and 2) to hair that has been treated with 200 mg of artificial sebum (3 and 4) at initial. Strand 1 was shampooed before color was applied whereas strand 2 was only rinsed with water. Strand 3 had sebum applied according to the foregoing protocol whereas strand 4 skipped the rinsing step. FIG. 4 shows the hair swatches of FIG. 3 after 20 washes with shampoo and conditioner. Longevity and initial color uptake are better for swatch 1 and 2 (no sebum), evidencing that artificial sebum application is a directional tool to mimic, at least partly, the challenges faced by coloring formulas on virgin hair or root.

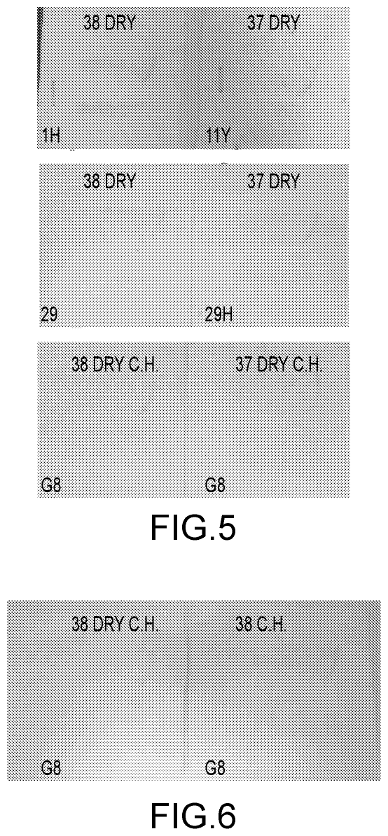

FIG. 5 shows a compilation of the rub-off test results for each timepoint when color gel 38 and 37A were applied to dry hair at low humidity. Less rub off was observed for the composition containing Si-BPEI resin.

Since more rub-off is typically observed for traditional hair color compositions at higher humidity, FIG. 6 shows rub-off of color gel 38A at higher humidity (23° C./45% RH) after 48 hours. Slight rub-off was observed when the color gel was applied to dry hair (left) whereas a more substantial rub-off was observed when the color gel was applied to wet hair (right). This this result was unexpected since the silicone compounds utilized are moisture curable.

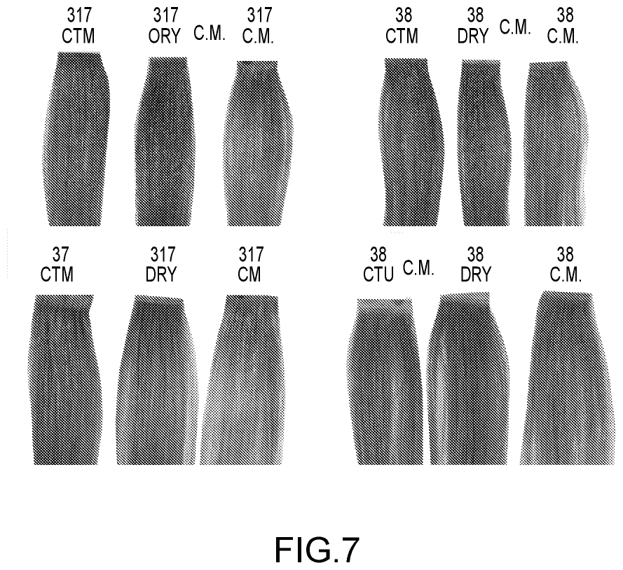

FIG. 7 shows a comparison of initial application and after 20 washes for color gel 38A containing BPEI to the control 37A (no BPEI) when the color was applied to wet or dry hair strands pre-treated with sebum. For low humidity (“LH”) samples, the color gels were applied to dry or wet hair strands and maintained at 16% RH. For high humidity (“CTH”) samples, the color gels were applied to wet hair strands and maintained at 45% RH. In all cases, formula 38A containing Si-BPEI visually improves initial color uptake and longevity. Color gel 38A showed a perceivable improvement of consistency across environmental conditions (relative humidity and temperature combinations and application methods, i.e., wet vs dry) compared to the control 37A. The incorporation of the BPEI in the resin can provide a significant advantage since its consistency across varying humidity and for wet or dry hair enables it to be used globally, in different environments and across seasons.

A comparison of initial application and after 20 washes was also performed for color gels 38A and 70A when the color was applied to dry hair strands pre-treated with sebum and maintained at 15% RH, 23° C. There was a noticeable improvement in rub off and a slight improvement in longevity for formula 70A containing GPTMS linking silane.

### Example 23—Stability of Silicone Resin Color Gel

The pot stability of silicone resin color gel 70A was tested up to 1 hour. The composition was applied to hair strands in the same manner as Example 22 immediately after mixing (0 min), then at 5, 10, 20, 30 and 60 minutes thereafter. The resin was cured for 50 minutes at ambient conditions, rinsed, conditioned, and blow dried. Then, 20 washing cycles were performed after 48 hours from color gel application.

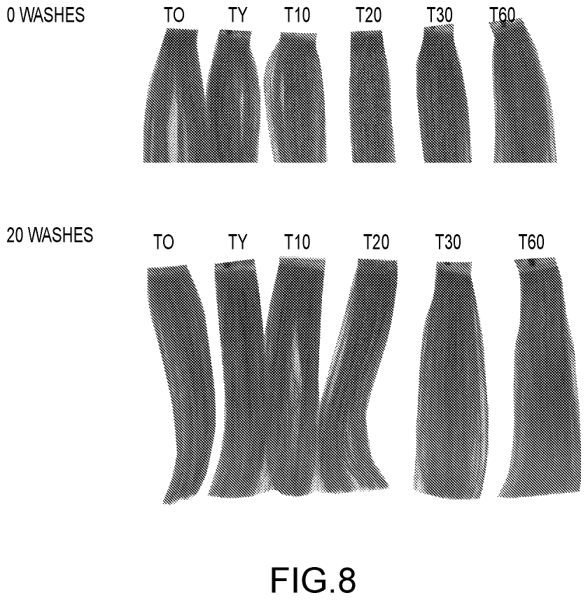

Pot life is an important parameter that ensures the final consumer will have sufficient time to apply the gel color to a full head without loss of performance due to the fast curing of the resin. FIG. 8 shows that consistent color was observed at each timepoint allowing up to 1 hour for a user to apply the color gel. The color intensity remained after 20 washing cycles at all timepoints tested.

### Example 24—Stability of Silicone Resins

The stability of the resin of Example 2 was tested by comparing separate batches. Silicone resin color gel 70A of Example 21 had been prepared after the resin of example 2 was stored for 2 months. A second resin according to Example 2 was prepared. Thereafter, the resin was stored for two weeks and used to prepare a silicone resin color gel 99A in accordance with the procedure set forth in Example 21. The silicone resin color gel 99A was applied to hair strands as set forth in Example 22. No difference was observed in rub-off or color retention for formula 70A and 99A.

### Example 25—Application of Silicone Resin Color Gel+Acrylate Sealer

Various acrylate sealer formulations were prepared by mixing 0.63 mmol of a selected acrylate, 0.05 g TPO-L, 1 g of a 30% aqueous solution of AQ—48U from Eastman, and 1 g of PP acrylate cellulose sealer (1% Natrosol, 1% Klucel, 10% Ecosmooth Satin P). The mixture was heated with a hair dryer for a few seconds and placed into an aluminum pan.

| Selected Acrylates | |
|---|---|
| (Sartomer) | Experimental Abbreviation |
| SR9038 | 9038 |
| SR454 | |
| SR499 | 499 |
| SR502 | 502 |
| SR9035 | |
| SR415 | 415 |

Hair swatches were pre-treated with sebum and silicone resin color gel 70A was applied to the hair swatches using the procedure set forth in Example 22 and the hair was cured for 10 minutes at 45% RH. The swatches were then immersed in the acrylate sealer mixture and rested for 3 minutes. Hair was rinsed with water and let to rest for 3 more minutes before exposing to UV light at 365 nm and air 1 minute per side. The exposed strands were rinsed, conditioned, and blow dried. Rub-offs and images were taken thereafter. After 48 hours, the strands were put through 20 washing cycles and images were taken. A comparison was made to strands in which no sealer was applied (N.S.) and strands to which only PP acrylate cellulose sealer was applied (R.S.). The comparators N.S. and R.S. were cured at 45% RH for 50 minutes.

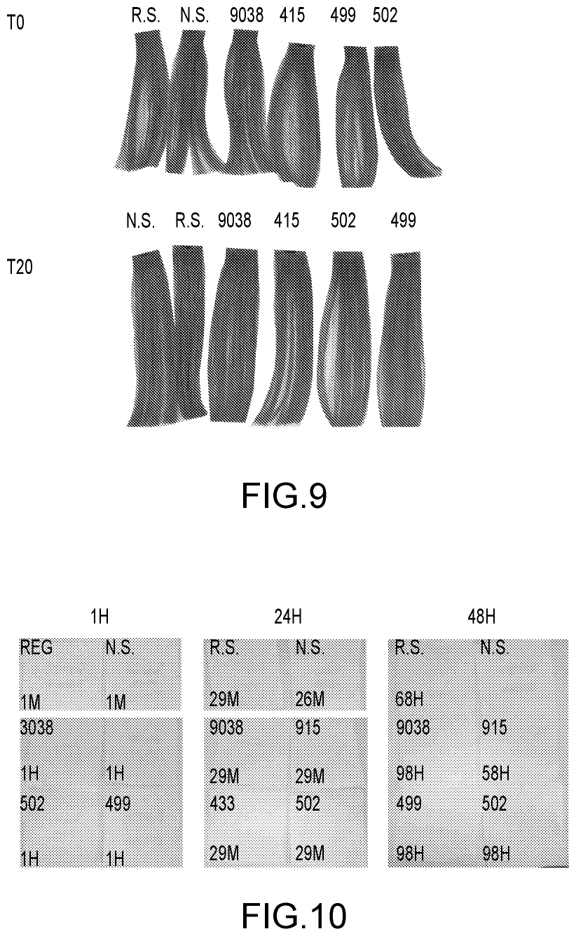

FIG. 9 shows images of the strands after initial treatment with sealer and after 20 washing cycles. No noticeable difference was observed.

FIG. 10 shows rub offs at 1, 24 and 48 hours. Hair sealed with a composition containing SR9038 and SR502 showed improved rub off results at 1 hour.

Use of UV-LED sealing curing in conjunction with the silicone resin cosmetic compositions disclosed herein can improve rub off hair initially colored with the resins and potentially decrease the time necessary to cure, but does not appear to improve the remanence of the color long term.

### Example 26—Synthesis of Silicone Resin

Methyltriethoxysilane (MTEOS) 23.89 g (134 mmol), water 1.44 g, HCl 0.5M 1.33 g, were added to a 100 mL 3 neck round bottom flask equipped with a condenser under magnetic stirring, and allowed to react at room temperature for 30 min. BPEI 600 1.068 g (2 mmol) was added and subsequently 3-glycidoxypropyltrimethoxysilane (GPTMS) 0.105 g (0.445 mmol) were added and the reaction was allowed to react for 2 h at room temperature, then 3-aminopropyltriethoxysilane (AMEO) 7.49 g (33.8 mmol) was added and the reaction was allowed to react for 30 min at room temperature, and then at 150° C. for 3 h. The resulting product was either packed out in a glass jar, or further concentrated by removal of the residual volatiles under reduced pressure at 80° C. for 1 h.

### Example 27—Synthesis of Silicone Resin

Methyltriethoxysilane (MTEOS) 45.395 (255 mmol), 1,8-bis(triethoxysilyl)octane 2.94 g (6.7 mmol), water 2.88, HCl 0.5M 2.66 g, were added to a 100 mL 3 neck round bottom flask equipped with a condenser under magnetic stirring, and allowed to react at room temperature for 30 min. BPEI 600 2.136 g (4 mmol) was added and subsequently 3-glycidoxypropyltrimethoxysilane (GPTMS) 0.210 g (1 mmol) were added and the reaction was allowed to react for 2 h at room temperature, then 3-aminopropyltriethoxysilane (AMEO) 14.98 g (67.7 mmol) was added and the reaction was allowed to react for 30 min at room temperature, and then at 90° C. for 1 h. The resulting product was either packed out in a glass jar, or further concentrated by removal of the residual volatiles under reduced pressure at 80° C. for 1h.

The moisture-curable silicone resins described herein containing silylated polyethyleneimine provide long lasting coatings to keratin fibers. Use of the resins can cut down on the number of steps in a hair treatment process by eliminating the need for a pretreatment, or to wet or dry hair to facilitate adhesion and curing. As a result, they provide a more environmentally friendly process for coloring hair.

It will be appreciated that, within the principles described by this specification, a number of variations exist. It should also be appreciated that the embodiments described are only embodiments, and are not intended to limit the scope, applicability, or construction of the claims in any way.

1. A cosmetic composition for coloring keratin fibers, the cosmetic composition comprising:
a moisture-curable silicone resin formed from a reaction of reactants comprising:
a silylated polyethyleneimine,
at least one hydrolyzable silane having the formula R¹ₙSiR²₄₋ₙ, wherein:
R¹ is alkyl, aryl, fluoroalkyl, trialkylsilyl, triarylsilyl, vinyl, or a combination thereof,
R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof,
n=0, 1, 2, or 3, and
R is alkyl or H; and
an acid or a base; and
at least one coloring agent selected from the group consisting of pigments, direct dyes, and a mixture thereof.

2. The cosmetic composition of claim 1, wherein the silylated polyethyleneimine has at least one amino functional group which is covalently bonded with a hydrolyzable silyl group —SiR²ₙ, wherein n=1, 2 or 3, from the at least one hydrolyzable silane.

3. The cosmetic composition of claim 1, wherein the reactants further comprise a hydrolyzable aminosilane having the formula R¹ₙSiR⁴₄₋ₙ, wherein:
R³ is aminoalkyl, aminoaryl, or a combination thereof,
R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof,
n=1, 2, or 3, and
R is alkyl or H.

4. The cosmetic composition of claim 3, wherein the hydrolyzable aminosilane is selected from the group consisting of 3-aminopropyltriethoxysilane, 3-aminopropyltrimethoxysilane, 11-aminoundecyltriethoxysilane, N1,N1-bis[3-triethoxylyl)propyl]-1,2 ethendiamine, N-3-[(amino(polypropylenoxy)amino-propyltrimethoxysilane, 3-aminopropyldiisopropylethoxysilane, bis(methyldiethoxysilylpropyl)amine, bis(triethoxysilylpropyl)amine, bis[3-trimethoxysilyl)propyl]-ethylenediamine, N-(2-aminoethyl)-11-aminoundecyl-trimethoxysilane, N-(3-acryloxy-2-hydroxypropyl)-3-aminopropyltriethoxysilane, N-(2-aminoethyl)-3-aminoisobutyldimethylmethoxysilane, N-(2-aminoethyl)-3-aminoisobutyl-methyldimethoxysilane, and combinations thereof.

5. The cosmetic composition of claim 1, wherein the at least one hydrolyzable silane having the formula R¹ₙSiR²₄₋ₙ comprises triethoxymethylsilane, methoxytrimethylsilane, dimethoxydimethylsilane, trimethoxymethylsilane, tetramethoxysilane, ethoxytrimethylsilane, diethoxydimethylsilane, tetraethoxysilane, vinyltrimethoxysilane, vinyltriethoxysilane, hexyltriethoxysilane, hexyltrimethoxysilane, n-octyltriethoxysilane, n-octyltrimethoxysilane, 1,8-bis(triethoxysilyl)octane, 1,8-bis(trimethoxysilyl)octane, n-dodecyltriethoxysilane, n-dodecyltrimethoxysilane, stearyl triethoxysilane, stearyltrimethoxysilane, and combinations thereof.

6. The cosmetic composition of claim 1, wherein the at least one hydrolyzable silane having the formula R¹ₙSiR²₄₋ₙ is tetraethoxysilane, triethoxymethylsilane, diethoxydimethylsilane, ethoxytrimethylsilane, n-octyltriethoxysilane, 1,8-bis(triethoxysilyl)octane, or a combination thereof.

7. The cosmetic composition of claim 1, wherein the silylated polyethyleneimine is a reaction product of reactants comprising polyethyleneimine and a hydrolyzable silane having the formula R⁵ₙSiR⁶₄₋ₙ, wherein:
R⁵ is epoxyalkyl, (meth)acryloxyalkyl, acryloxyalkyl, halogenalkyl, isocyantoalkyl, or a combination thereof,
R⁶ is alkoxy, aryloxy, acetoxy, oximino, enoxy, amino, H, OH, halogen, α-hydroxycarboxylic acid amide (—OCR₂CONR₂), α-hydroxycarboxylic acid ester (—OCR₂COOR), or a combination thereof,
n=1, 2, or 3, and
R is alkyl or H.

8. The cosmetic composition of claim 7, wherein a molar ratio of the hydrolyzable silane having the formula R⁵ₙSiR⁶₄₋ₙ to the polyethyleneimine is from about 1:100 to about 100:1.

9. The cosmetic composition of claim 7, wherein the hydrolyzable silane having the formula R⁵ₙSiR⁶₄₋ₙ is selected from the group consisting of 3-methacryloxypropyltriethoxysilane, 3-acryloxypropyltriethoxysilane, 3-glycidoxypropyltrimethoxysilane, 3-glycidoxypropyltriethoxysilane, 3-isocyanatopropyltriethoxysilane, 3-methacryloxypropyltrimethoxysilane, 3-acryloxypropyltrimethoxysilane, 3-isocyanatopropyltrimethoxysilane, 3-chloropropyltrimethoxysilane, 3-chloropropyltriethoxysilane, and combinations thereof.

10. The cosmetic composition of claim 7, wherein the polyethyleneimine is a linear, a branched, or a dendrimer polyethyleneimine having a molecular weight ranging between 300 and 100,000.

11. The cosmetic composition of claim 1, wherein the reactants further comprise at least one agent selected from the group consisting of plasticizers, vitamins, fragrances, trace elements, softeners, plasticizers, coalescers, preserving agents, stabilizers, co-resins, anti-foams, spreading agents, and combinations thereof.

12. A kit for forming the cosmetic composition of claim 1, the kit comprising:
a first container containing the moisture-curable silicone resin: and
a second container containing the at least one coloring agent.

13. The kit of claim 12, further comprising a third container containing a sealer.

14-16. (canceled)

17. A cosmetic composition for coloring keratin fibers, the cosmetic composition comprising:
a moisture-curable silicone resin formed from a reaction of reactants comprising:
at least one hydrolyzable silane having the formula R¹ₙSiR²₄₋ₙ, wherein:
R¹ is alkyl, aryl, fluoroalkyl, trialkylsilyl, triarylsilyl, vinyl, or a combination thereof,
R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof, and
n=0, 1, 2, or 3;
a hydrolyzable aminosilane having the formula R³ₙSiR⁴₄₋ₙ, wherein:
R³ is aminoalkyl, aminoaryl, or a combination thereof,
R⁴ is alkoxy, aryloxy, acetoxy, oximino, enoxy, amino, aminoalkoxy, α-hydroxycarboxylic acid amide (—OCR₂CONR₂), α-hydroxycarboxylic acid ester (—OCR₂COOR), H, or a combination thereof, and
n=1, 2, or 3;
a hydrolyzable silane having the formula R⁵ₙSiR⁶₄₋ₙ, wherein:
R⁵ is epoxyalkyl, methacryloxyalkyl, acryloxyalkyl, haloalkyl, or a combination thereof,
R⁶ is alkoxy, aryloxy, acetoxy, oximino, enoxy, amino, amino, H, OH, halogen, α-hydroxycarboxylic acid amide (—OCR₂CONR₂), α-hydroxycarboxylic acid ester (—OCR₂COOR), or a combination thereof, and
n=1, 2, or 3;
polyethyleneimine; and
an acid or a base; and
at least one coloring agent selected from the group consisting of pigments, direct dyes, and a mixture thereof.

18. The cosmetic composition of claim 17, wherein a molar ratio of the hydrolyzable silane having the formula R⁵ₙSiR⁶₄₋ₙ to the polyethyleneimine is from about 1:100 to about 100:1.

19. A kit for forming the cosmetic composition of claim 17, the kit comprising:
a first container containing the moisture-curable silicone resin; and
a second container containing the at least one coloring agent.

20. The kit of claim 19, further comprising a third container containing a sealer.

21. A method of coloring keratin fibers, the method comprising:
applying a composition comprising:
a moisture-curable silicone resin formed from a reaction of reactants comprising:
polyethyleneimine;
at least one hydrolyzable silane having the formula R¹ₙSiR²₄₋ₙ, wherein:
R¹ is alkyl, aryl, fluoroalkyl, trialkylsilyl, triarylsilyl, vinyl, or a combination thereof,
R² is alkoxy, aryloxy, acetoxy, oximino, enoxy, α-hydroxycarboxylic acid amide (—OCR₂CONR₂), α-hydroxycarboxylic acid ester (—OCR₂COOR), H, halogen, or a combination thereof,
n=0, 1, 2, or 3, and
R is alkyl or H;
a hydrolyzable silane having the formula R⁵ₙSiR⁶₄₋ₙ, wherein:
R⁵ is epoxyalkyl, (meth)acryloxyalkyl, acryloxyalkyl, haloalkyl, or
a combination thereof,
R⁶ is alkoxy, aryloxy, acetoxy, oximino, enoxy, amino, amino, H, OH, halogen, α-hydroxycarboxylic acid amide (—OCR₂CONR₂), α-hydroxycarboxylic acid ester (—OCR₂COOR), or a combination thereof, and
n=1, 2, or 3; and
an acid or a base;
at least one coloring agent selected from the group consisting of pigments, direct dyes, or a mixture thereof, and
at least one agent selected from the group consisting of plasticizers, vitamins, fragrances, trace elements, softeners, plasticizers, coalescers, preserving agents, stabilizers, co-resins, anti-foams, spreading agents, and a combination thereof.

22. The method of claim 21, further comprising applying a sealer to the keratin fibers after applying the composition.

23. The method of claim 22, further comprising curing the sealer with UV-LED light.

24-26. (canceled)
